(19) United States
(12) Patent Application Publication
(10) Pub. No.: US 2018/0071008 A1
(43) Pub. Date: Mar. 15, 2018

(54) INSTRUMENTS AND METHODS FOR THERMAL TISSUE TREATMENT

(71) Applicant: ElectroMedical Associates LLC, Bethesda, MD (US)

(72) Inventors: Robert A. VAN WYK, St. Pete Beach, FL (US); Yuval CARMEL, Rockville, MD (US); Anatoly SHKVARUNETS, Rockville, MD (US)

(73) Assignee: ElectroMedical Associates LLC, Bethesda, MD (US)

(21) Appl. No.: 15/815,010

(22) Filed: Nov. 16, 2017

Related U.S. Application Data
(63) Continuation-in-part of application No. 13/905,774, filed on May 30, 2013, now Pat. No. 9,827,033.
Application No. 13/905,774 is a division of application No. 12/033,987, filed on Feb. 20, 2008, now Pat. No. 8,475,452.
(60) Provisional application No. 60/902,548, filed on Feb. 21, 2007.

(51) Int. Cl. A61B 18/08; A61B 18/18

(52) U.S. Cl. CPC A61B 18/082; A61B 18/18; A61B 2018/1472; A61B 2218/002

(57) ABSTRACT

Disclosed herein are high efficiency surgical devices and methods of using same using radio frequency (RF) electrical power to destroy tumors, form lesions, denaturize, desiccate, coagulate and ablate soft tissues, as well as to drill, cut, resect and vaporize soft tissues. More particularly, the electrosurgical instruments use externally supplied conductive or non-conductive liquids, delivered to an area of interest in the form of heated irrigant and/or steam, to thermally treat target tissues of interest, either at the tissue surface, below the tissue surface or at a site remote therefrom.

### PRIORITY

This application is a continuation-in-part of U.S. patent application Ser. No. 13/905,774 filed May 30, 2013, now U.S. Pat. No. 9,827,033 issued Nov. 28, 2017, which, in turn, is a division of U.S. patent application Ser. No. 12/033,987 filed Feb. 20, 2008, now U.S. Pat. No. 8,475,452 issued Jul. 2, 2013, which, in turn, claims the benefit of U.S. Provisional Application No. 60/902,548 filed Feb. 21, 2007. These prior applications are incorporated by reference herein in their entirety.

### FIELD OF THE INVENTION

The present invention relates generally to the field of thermal tissue treatment, and more particularly, to high efficiency surgical instruments and methods that use radio frequency (RF) electrical power to destroy tumors, form lesions, denaturize, desiccate, coagulate and ablate soft tissues, as well as to drill, cut, resect and vaporize soft tissues. More particularly, the electrosurgical instruments of the present invention use externally supplied conductive or non-conductive fluids, delivered to an area of interest in the form of heated irrigant and/or steam, to thermally treat target tissues of interest, either at the tissue surface, below the tissue surface or at a site remote therefrom.

### BACKGROUND OF THE INVENTION

Electrosurgical procedures are advantageous since they generally reduce patient bleeding and trauma. The devices used are electrically energized, typically using an RF generator operating at a frequency that ranges between 100 kHz to over 4 MHz. Due to their ability to provide beneficial outcomes with reduced patient pain and recuperation time, electrosurgical devices have recently gained significant popularity recently. In common terminology and as used herein, the term “electrode” can refer to one or more components of an electrosurgical device (such as an “active electrode” or a “return electrode”) or to the entire device, as in an “ablator electrode” or “cutting electrode”. Electrosurgical devices may also be referred to as electrosurgical “probes” or “instruments”.

Many types of electrosurgical instruments are currently in use, and can be divided into two general categories: monopolar devices and bipolar devices. In the context of monopolar electrosurgical devices, the RF current generally flows from an exposed active electrode, through the patient's body, to a passive, return current electrode that is externally attached to a suitable location on the patient body. In this manner, the patient's body becomes part of the return current circuit. In the context of bipolar electrosurgical devices, both the active and the return current electrodes are exposed, and are typically positioned in close proximity to each other, more frequently mounted on the same instrument. The RF current flows from the active electrode to the return electrode through the nearby tissue and conductive fluids.

The need to effectively yet minimally invasively treat tumor tissue from a patient's body arises in the context of many medical practice areas, including, but not limited to, oncology, ear nose and throat (ENT), urology, gynecology, laparoscopy and general surgery. More specifically, there is often a need to denaturize, desiccate or coagulate tissue and destroy tumors in the liver, kidney, breast, lung, bone, lymph nodes, nerve ganglia and other organs. Such procedures are collectively referred to as tissue ablation or lesion formation, and are often used to destroy tumors without radical surgery. In such cases, an effective treatment is one in which the tumor itself, and perhaps a small margin of tissue around the tumor, is affected. The affected tumor tissue is not immediately removed. Over time, the dead tissue will naturally shrink, dissolve and, in some cases, be gradually replaced by scar tissue.

Although the benefits of these procedures are well recognized by those of skill in the art, current electrosurgical instruments and procedures suffer from very significant deficiencies. Quite often existing instruments are composed of one or more needles which are electrically energized by radiofrequency. As a result, the energy deposition in the tissue is concentrated close to where the needle is positioned, leading to overheating in the immediate region and under-heating in areas farther away. The result is a highly non-homogeneous energy deposition and highly non-homogeneous lesion. It is inherently impossible to accurately control the shape and size of the lesion formed with existing instruments because the energy deposition and heating occurs from the inside out. However, in order to destroy a tumor, it is often necessary, yet undesirable, to also destroy a large margin of healthy tissue around the tumor. As a result, the current processes are inefficient, require high power levels and therefore can lead to unnecessary complications and undesired side effects. In some cases, additional return electrodes (also called grounding pads or patient electrodes) are needed in order to safely handle the high energy and high current required to perform the procedure. One such system marketed by Boston Scientific (Natick, Mass.) for liver ablation uses four patient electrodes simultaneously.

In view of these and other deficiencies, there is a need in the art for improved electrosurgical instruments that are capable of creating uniform lesions of a desired size and shape, capable of treating tissue and tumors from the outside in rather or from the inside out, and capable of treating large and non-uniform tumors and leaving healthy tissue unharmed. There is also a need in the art for a high efficiency electrosurgical instrument capable of destroying the tumor at relatively low power, thereby increasing patient safety and efficacy and reducing undesired side effects.

### SUMMARY OF THE INVENTION

It is accordingly an object of the present invention to provide highly efficient, minimally invasive surgical instruments capable of overcoming the deficiencies discussed above. More particularly, in view of the ever-present need in the art for improvements in electrode design, it is an object of the present invention to provide highly efficient and efficacious electrosurgical instruments suitable for the thermal treatment of tumors, more particularly radiofrequency electrosurgical devices adapted for enhanced lesion formation.

Electrosurgical instruments of the present invention may be designed to be inserted directly, to penetrate the patient tissue at the desired location, or alternatively to be introduced into the patient body through a cannula, a resectoscope, an endoscope or an opening in the body.

In certain embodiments, the electrosurgical instruments of the present invention may optionally be provided with means for externally supplying electrically conductive or non-conductive irrigation liquid to the surgical site. In other embodiments, the irrigation fluid may be stored in the instrument itself or in the handpiece. In other embodiments, the electrosurgical instrument of the present invention may be designed to function in the absence of an external source of fluids, relying instead on the tissue properties or endogenous bodily fluids. As noted above, this mode of operation is sometimes referred to as “dry field”.

In further embodiments, the electrosurgical instrument of the present invention may optionally be equipped with irrigation, aspiration or both.

The electrosurgical instrument of the present invention may be either monopolar or bipolar electrodes and may optionally be equipped with one or more floating elements. “Floating” electrodes for electrosurgery are described in U.S. Pat. Nos. 7,563,261 and 7,566,333, the contents of which are incorporated by reference herein in their entirety. As discussed in detail therein, the floating electrode has a potential that is between the potential of the active electrode and the return electrode. The presence of the floating electrode can strongly, and beneficially, influence the distribution on the induced current and electric field in, and in the vicinity, of the treatment zone (a process referred to as “focusing”).

It will be understood by those skilled in the art that one or more aspects of this invention can meet certain objectives, while one or more other aspects can meet certain other objectives. Each objective may not apply equally, in all its respects, to every aspect of this invention. As such, the following objects should be viewed in the alternative with respect to any one aspect of this invention:

Thus, it is an object of the present invention to provide an electrosurgical instrument for thermal tissue treatment composed of:
(a) an elongate shaft having a proximal end configured for connection to a power source and a distal end having an electrode assembly mounted thereto;
(b) an electrode assembly comprising an active electrode, a floating electrode, and an insulator separating the active and floating electrodes and defining a cavity therebetween;
(c) a means for supplying an irrigant to the cavity;

wherein the insulator is formed from a nonconductive dielectric material while said active and floating electrodes are formed from an electrically conductive material;

wherein the active and floating electrodes are positioned in close proximity to each other;

wherein the active electrode is connected via conductive means, such as cabling, disposed within said shaft to said power source while the floating electrode is not connected to a power source such that powering of the active electrode results in flow of current from the active electrode to said floating electrode via the irrigant, thereby resulting in the heating of the irrigant and the generation of steam;

wherein the heated irrigant and steam contacts target tissue so as to thermally treat the target tissue of interest.

It is a further object of the present invention to provide an electrosurgical instrument for sub-surface thermal treatment of target tissue composed of:
(a) an elongate shaft having a proximal end configured for connection to a power source and a distal end having an electrode assembly mounted thereto;
(b) an electrode assembly including (i) an insulating tubular member, (ii) an active electrode disposed at the distal tip of the insulating tubular member and connected via cabling disposed within the shaft to said power source, and (iii) a tubular conductive member concentrically disposed about the insulating tubular member; and
(c) optionally, a switching means for alternately connecting and disconnecting the conductive member to a power source;

wherein the insulating tubular member is formed from a nonconductive dielectric material while the active electrode and said conductive member are formed from an electrically conductive material;

wherein the active and floating electrodes are positioned in close proximity to each other but are prevented by the insulator from directly contacting each other; and

wherein the floating electrode takes the form of a tapered conical member that is sufficiently sharp to permit insertion of the electrode assembly into the target tissue.

It is yet a further object of the present invention to provide a method for thermally treating a target tissue in the body of a patient including the steps of:
(a) introducing an electrosurgical instrument according to the principles of the present invention into the patient such that the electrode assembly is in close contact with the target tissue;
(b) supplying an irrigant to the cavity defined between the active and floating electrodes; and
(c) applying a high-frequency voltage to the active electrode;

wherein the high frequency voltage results not only in the flow of current among active electrode, floating electrode and target tissue but further results in the boiling of irrigant, such that expanding steam and heated irrigant flow from the cavity to the target tissue site, thereby thermally treating the target tissue.

The present invention relates generally to the field of thermal tissue treatment, and more particularly, to high efficiency surgical instruments and methods which use radio frequency (RF) electrical power and/or electrically heated filaments to destroy tumors, form lesions, denaturize, desiccate, coagulate and ablate soft tissues, as well as to drill, cut, resect and vaporize soft tissues. According to the principles of this invention, the surgical instruments of the present invention can be used with externally supplied conductive or non-conductive liquids, as well as without externally supplied liquids, a mode of operation often referred to as “dry field” environment.

In one embodiment, the present invention provides a high efficiency electrosurgical instrument particularly suited to surface treatment of tissues, such a tumor tissues, the instrument including an active end having radiused corners and composed of a unique combination of active electrode, insulator, floating electrode and return electrode that limit sparking and tissue vaporization. Illustrative examples of this object are set forth in FIGS. 1-22.

In another embodiment, the present invention provides a high efficiency electrosurgical instrument wherein the active electrode and floating electrode interact to boil an exogenous irrigant therebetween such that lesion formation is accomplished primarily by steam and heated fluid which contact the tissue. Illustrative examples of this object are set forth in FIGS. 23-29.

In yet another embodiment, the present invention provides a high efficiency electrosurgical instrument particularly suited to sub-surface tissue treatment, the instrument including a switching means that allows a circumferential electrode to function as a floating electrode when drilling into the tissue, and subsequently as an active electrode to thermally treat tissue when in close proximity to a target site. Illustrative examples of this object are set forth in FIGS. 30-32.

In a further embodiment, the present invention provides a high efficiency electrosurgical instrument particularly suited to sub-surface tissue treatment, wherein the instrument uses heated irrigant and steam generated within the probe to thermally treat tissue in close proximity. In one embodiment, the heating occurs within the instrument tip, between an active tip electrode and a floating electrode in contact with the tissue. Illustrative examples of this object are set forth in FIGS. 33-58.

In a still further embodiment, the present invention provides a high efficiency electrosurgical instrument particularly suited to treating tissue from one or more adjacent surfaces, wherein the instrument uses heated irrigant and steam generated within the probe to treat tissue in close proximity. Illustrative examples of this object are set forth in FIGS. 59-63.

These and other objects and features of the invention will become more fully apparent when the following detailed description is read in conjunction with the accompanying figures and/or examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art having knowledge of electrode design. Such objects, features, benefits and advantages will be apparent from the above in conjunction with the accompanying examples, data, figures and all reasonable inferences to be drawn there-from, alone or with consideration of the references incorporated herein.

### BRIEF DESCRIPTION OF THE DRAWINGS

Various aspects and applications of the present invention will become apparent to the skilled artisan upon consideration of the brief description of the figures and the detailed description of the present invention and its preferred embodiments which follows:

FIG. 1 is a plan view of an insulator for a lesion forming electrosurgical instrument formed in accordance with the principles of this invention.

FIG. 2 is a side elevational view of the objects of FIG. 1.

FIG. 3 is a bottom side plan view of the objects of FIG. 1.

FIG. 4 is a perspective view of the objects of FIG. 1.

FIG. 5 is an axial sectional view of the objects of FIG. 1 at location A —A of FIG. 1.

FIG. 6 is a side elevational sectional view of the objects of FIG. 1 at location B-B of FIG. 1.

FIG. 7 is a plan view of an active electrode for a lesion forming electrosurgical instrument formed in accordance with the principles of this invention.

FIG. 8 is a side elevational view of the objects of FIG. 7.

FIG. 9 is a perspective view of the objects of FIG. 7.

FIG. 10 is a distal axial view of the objects of FIG. 7.

FIG. 11 is a plan view of a floating electrode for a lesion forming electrosurgical instrument formed in accordance with the principles of this invention.

FIG. 12 is a side elevational view of the objects of FIG. 11.

FIG. 13 is a bottom side plan view of the objects of FIG. 11.

FIG. 14 is a perspective view of the objects of FIG. 11.

FIG. 15 is an axial view of the objects of FIG. 11.

FIG. 16 is a plan view of a distal portion of a lesion forming electrosurgical instrument formed in accordance with the principles of this invention.

FIG. 17 is a side elevational view of the objects of FIG. 16.

FIG. 18 is a bottom side plan view of the objects of FIG. 16.

FIG. 19 is a perspective view of the objects of FIG. 16.

FIG. 20 is an axial sectional view of the objects of FIG. 16 at location C-C of FIG. 17.

FIG. 21 is a side elevational sectional view of the objects of FIG. 16 at location D-D of FIG. 16.

FIG. 22 is an axial sectional view of the objects of FIG. 16 during use showing current flow.

FIG. 23 is a plan view of a distal portion of an alternate embodiment electrosurgical instrument adapted for thermal tissue treatment near a tissue surface.

FIG. 24 is a side elevational view of the objects of FIG. 23.

FIG. 25 is a bottom side plan view of the objects of FIG. 23.

FIG. 26 is a side elevational sectional view of the objects of FIG. 23 at location B-B of FIG. 23.

FIG. 27 is an axial sectional view of the objects of FIG. 23 at location E-E of FIG. 24.

FIG. 28 is an axial sectional view of the objects of FIG. 23 in use in a dry field environment.

FIG. 29 is an axial sectional view of the objects of FIG. 23 in use in a conductive fluid environment.

FIG. 30 is a perspective view of a distal portion of an alternate embodiment RF electrosurgical instrument adapted for thermal tissue treatment at a location remote from the tissue surface (e.g., sub-surface).

FIG. 31 is a plan view of the objects of FIG. 30.

FIG. 32 is a side elevational sectional view of the objects of FIG. 30 at location A —A of FIG. 31.

FIG. 33 is a perspective view of a distal portion of an alternate embodiment RF electrosurgical instrument adapted for thermal tissue treatment at a location remote from the tissue surface (e.g., sub-surface).

FIG. 34 is a plan view of the objects of FIG. 33.

FIG. 35 is a side elevational sectional view of the objects of FIG. 34 at location A —A of FIG. 34.

FIG. 36 is a sectional view of the objects of FIG. 33 during use.

FIG. 37 is a perspective view of a radio frequency device of the present invention for the thermal treatment of tissue.

FIG. 38 is an expanded view of the objects of FIG. 37 at location C.

FIG. 39 is a plan view of the objects of FIG. 37.

FIG. 40 is a side elevational view of the objects of FIG. 37.

FIG. 41 is an expanded view of the objects of FIG. 39 at location A.

FIG. 42 is an expanded view of the objects of FIG. 40 at location B.

FIG. 43 is a sectional view of the objects of FIG. 41 at location A-A.

FIG. 44 is an expanded view of the distal portion of the objects of FIG. 43 during use depicting the inflow of irrigant, it conversion to steam, and the flow of this steam to the distal portion of the probe and therefrom to tissue in proximity.

FIG. 45 is an expanded view of the distal portion of the objects of FIG. 43 during use depicting the flow of RF energy through the device to tissue in proximity.

FIG. 46 is a sectional view of the distal portion of an alternate embodiment thermal treatment device of the present invention.

FIG. 47 is a sectional view of the distal portion of another alternate embodiment thermal treatment device of the present invention.

FIG. 48 is a sectional view of the distal portion of another alternate embodiment thermal treatment device of the present invention.

FIG. 49 is a sectional view of the distal portion of another alternate embodiment thermal treatment device of the present invention.

FIG. 50 is an expanded view of the objects of FIG. 49 at location A.

FIG. 51 is a sectional view of the distal portion of another alternate embodiment thermal treatment device of the present invention.

FIG. 52 is an expanded view of the objects of FIG. 51 at location A.

FIG. 53 is a distal perspective view of an alternate embodiment thermal treatment device of the present invention.

FIG. 54 is an expanded view of the objects of FIG. 53 at location B.

FIG. 55 is a plan view of the objects of FIG. 53.

FIG. 56 is an expanded view of the objects of FIG. 55 at location A.

FIG. 57 is a sectional view of the objects of FIG. 56 at location A-A.

FIG. 58 is an expanded view of the objects of FIG. 57 at location C.

FIG. 59 is a sectional view of the distal portion of an alternate embodiment of the present invention.

FIG. 60 is an expanded view of the objects of FIG. 59 at location B.

FIG. 61 depicts the device of FIGS. 59 and 60 in use thermally treating tissue in proximity to a tissue surface.

FIG. 62 is an expanded view of the objects of FIG. 61 at location B.

FIG. 63 depicts thermal treatment of a tissue portion cooperatively using two of the distal portions of FIGS. 59 and 60.

FIG. 64 schematically depicts an adaptor circuit for a thermal treatment system of the present invention.

FIG. 65 schematically depicts an alternate embodiment adaptor circuit for a thermal treatment system of the present invention.

FIG. 66 is a sectional view of the distal portion of an alternate embodiment bipolar thermal treatment device of the present invention.

FIG. 67 is an expanded view of the objects of FIG. 66 at location A.

FIG. 68 schematically depicts a thermal treatment system of the present invention with a first adaptor circuit connected to the bipolar device of FIG. 66.

FIG. 69 schematically depicts a thermal treatment system of the present invention with a second adaptor circuit connected to the bipolar device of FIG. 66.

### DETAILED DESCRIPTION OF THE PREFERRED EMBODIMENTS

This present invention constitutes a marked improvement in the field of electrosurgery, more particularly, to high efficiency electrosurgical surgical instruments and methods which use radio frequency (RF) electrical power to destroy tumors, form lesions, denaturize, desiccate, coagulate and ablate soft tissues, as well as to drill, cut, resect and vaporize soft tissues.

Although any methods and materials similar or equivalent to those described herein can be used in the practice or testing of embodiments of the present invention, the preferred methods, devices, and materials are now described. However, before the present materials and methods are described, it is to be understood that this invention is not limited to the particular compositions, methodologies or protocols herein described, as these may vary in accordance with routine experimentation and optimization. It is also to be understood that the terminology used in the description is for the purpose of describing the particular versions or embodiments only, and is not intended to limit the scope of the present invention which will be limited only by the appended claims.

### Elements of the Present Invention

In the context of the present invention, the following definitions apply:

The words “a”, “an”, and “the” as used herein mean “at least one” unless otherwise specifically indicated.

In common terminology and as used herein, the term “electrode” may refer to one or more components of an electrosurgical device (such as an active electrode or a return electrode) or to the entire device, as in an “ablator electrode” or “cutting electrode”. Such electrosurgical devices are often interchangeably referred to herein as electrosurgical “probes” or “instruments”.

The present invention makes reference to an “active electrode” or “active element”. As used herein, the term “active electrode” refers to one or more conductive elements formed from any suitable metallic material, such as stainless steel, nickel, titanium, tungsten, and the like, connected, for example via cabling disposed within the elongated proximal portion of the instrument, to a power supply, for example, an externally disposed electrosurgical generator, and capable of generating an electric field.

The present invention makes reference to a “floating electrode” or “floating element”. As used herein, the term “floating electrode” refers to one or more conductive elements formed from any suitable metallic material, such as stainless steel, nickel, titanium, tungsten, and the like, that while disconnected from any power are nevertheless capable of intensifying the electric field in proximity to the active electrode and aid in bubble retention when the instrument is used to vaporize tissue.

The present invention makes reference to a “return electrode”. As used herein, the term “return electrode” refers to one or more powered conductive elements on the instrument formed from any suitable metallic material, such as stainless steel, nickel, titanium, tungsten, and the like, to which current flows after passing from the active electrode(s) and through the plasma field, or to a passive, return current electrode that is externally attached to a suitable location on the patient body.

The term “proximal” refers to that end or portion which is situated closest to the user; in other words, the proximal end of an electrosurgical instrument of the instant invention will typically include the handle portion.

The term “distal” refers to that end or portion situated farthest away from the user; in other words, the distal end of an electrosurgical instrument of the instant invention will typically include the active electrode portion.

The present invention makes reference to the thermal treatment of tissue, more preferably soft tissue, even more preferably tumor tissue. As used herein, the term “tissue” refers to biological tissues, generally defined as a collection of interconnected cells that perform a similar function within an organism. Four basic types of tissue are found in the bodies of all animals, including the human body and lower multicellular organisms such as insects, including epithelium, connective tissue, muscle tissue, and nervous tissue. These tissues make up all the organs, structures and other body contents. The present invention is not limited in terms of the tissue to be treated but rather has broad application to the thermal treatment of any target tissue with particular applicability to the ablation, removal and/or destruction of benign and cancerous tumors.

The instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal.

Unless otherwise defined, all technical and scientific terms used herein have the same meaning as commonly understood by one of ordinary skill in the art to which this invention belongs. In case of conflict, the present specification, including definitions, will control.

### Utilities of the Present Invention

As noted above, the present invention is directed to high efficiency monopolar or bipolar electrosurgical instruments and methods which utilize radio frequency (RF) energy, electrically energized filaments, and/or non-coherent radiation emitted by heated filaments to destroy tumors, form lesions, denaturize, desiccate, coagulate and ablate soft tissues, as well as to drill, cut, resect and vaporize soft tissues, with or without externally supplied liquids, having particular utility in the context of oncology, ear nose and throat (ENT), urology, gynecology, and laparoscopy, as well as general surgery.

Certain embodiments of the electrosurgical instrument of the present invention find particular utility in the treatment of tissue surfaces. Others are configured for sub-surface tissue treatment. Similarly, while some embodiments utilize the endogenous fluid of a “wet field” environment to transmit current to target sites, others require an exogenous irrigant. In certain embodiments, the irrigant is heated to the boiling point, whereby thermal tissue treatment arises through direct contact with either the boiling liquid itself or steam associated therewith.

As described in further detail below, in one aspect, the present invention expands on the floating electrode concept. For example, the present invention relates to the design and deployment of novel “floating electrode” electrosurgical instruments that use steam/hot fluid to thermally treat target tissue, both at the surface and below the surface.

The tissue treatment instruments of the present invention may be used in conjunction with existing diagnostic and imaging technologies, for example imaging systems including, but not limited to, MRI, CT, PET, x-ray, fluoroscopic, and ultrasound. Such imaging technology may be used to monitor the introduction and operation of the instruments of the present invention. For example, existing imaging systems may be used to determine location of target tissue, to confirm accuracy of instrument positioning, to assess the degree of thermal tissue treatment (e.g., sufficiency of tissue removal), to determine if subsequent procedures are required, and to assist in the atraumatic removal of the instrument.

As noted above, the instruments of the present invention find utility in thermal tissue treatment, more particularly in thermal treatment of tumor tissue, both benign and cancerous, to destroy tumors, form lesions, denaturize, desiccate, coagulate and/or ablate tumor tissues, as well as to drill, cut, resect and vaporize tumor tissues, with or without externally supplied liquids. Though the present invention is not particularly limited to the treatment of any one specific disease or the removal of any one specific type of tumor, the instruments of the present invention nevertheless find particular utility in the treatment and removal of liver, breast, bladder and spinal tumors, uterine fibroids, ovarian cysts, and colon polyps as well as the treatment of noncancerous conditions such as endometriosis.

### Illustrative Embodiments of the Present Invention

Hereinafter, the present invention is described in more detail by reference to the exemplary embodiments. However, the following examples only illustrate aspects of the invention and in no way are intended to limit the scope of the present invention. As such, embodiments similar or equivalent to those described herein can be used in the practice or testing of the present invention.

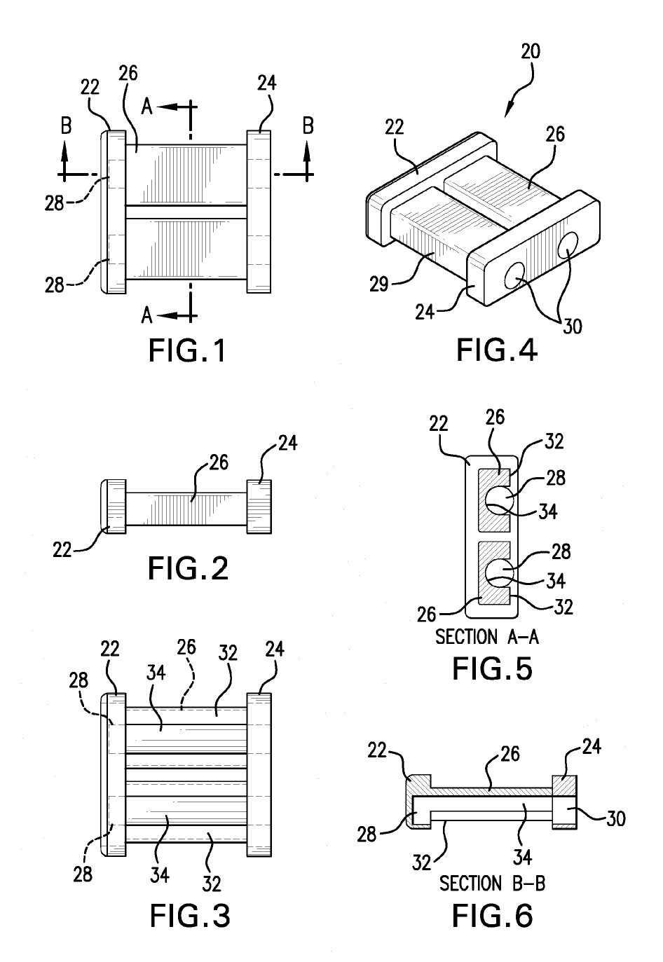

Referring to FIGS. 1 through 6, which depict an insulator of an electrosurgical instrument of the present invention that is particularly suited to thermally treating patient tissue, insulator 20 has a distal end portion 22, a proximal end portion 24 and mid-portions 26. Distal end portion 22 has formed in its proximal face cylindrical recesses 28. Proximal end portion 24 has axial cylindrical openings 30 axially aligned with recesses 28. Mid-portions 26 have formed in lower surface 32 channels 34. Insulator 20 is made from a suitable dielectric material, examples of which include, but are not limited to, alumina, zirconia, and high-temperature polymers.

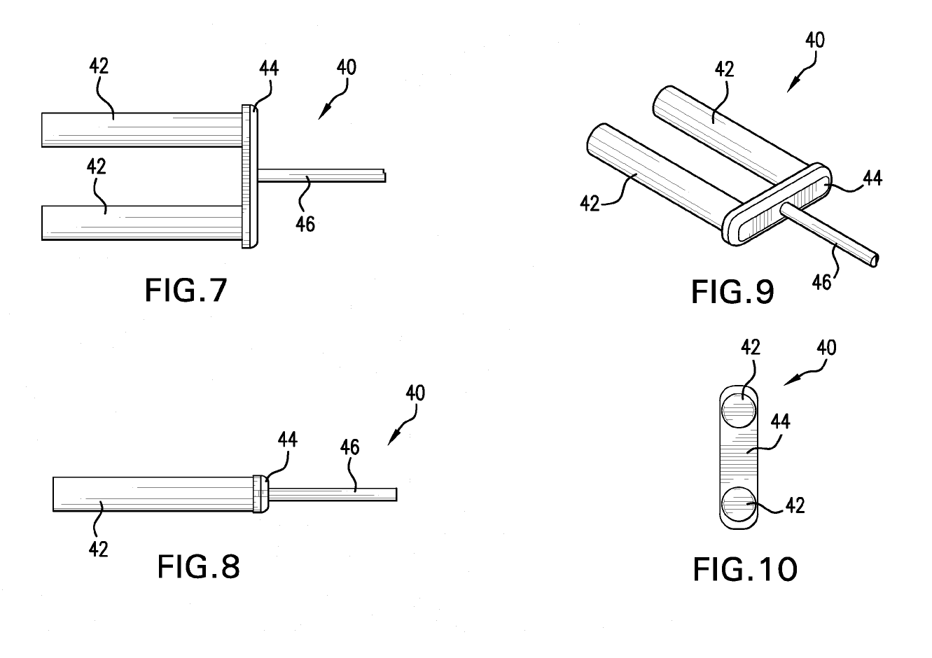

Referring to FIGS. 7 through 10, which depict an active electrode of an electrosurgical instrument of the present invention that is particularly suited to thermally treating patient tissue, active electrode 40 has a distal portion forming parallel cylindrical portions 42 connected by flange 44 to proximal conductor 46. Electrode 40 may be formed from any suitable metallic material, examples of which include, but are not limited to, stainless steel, nickel, titanium, tungsten, and the like.

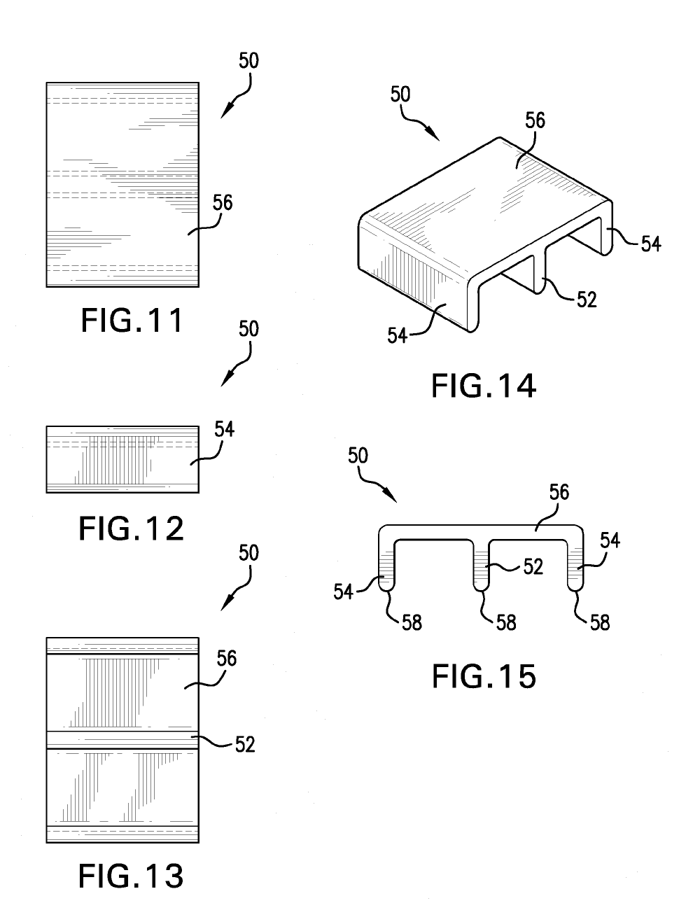

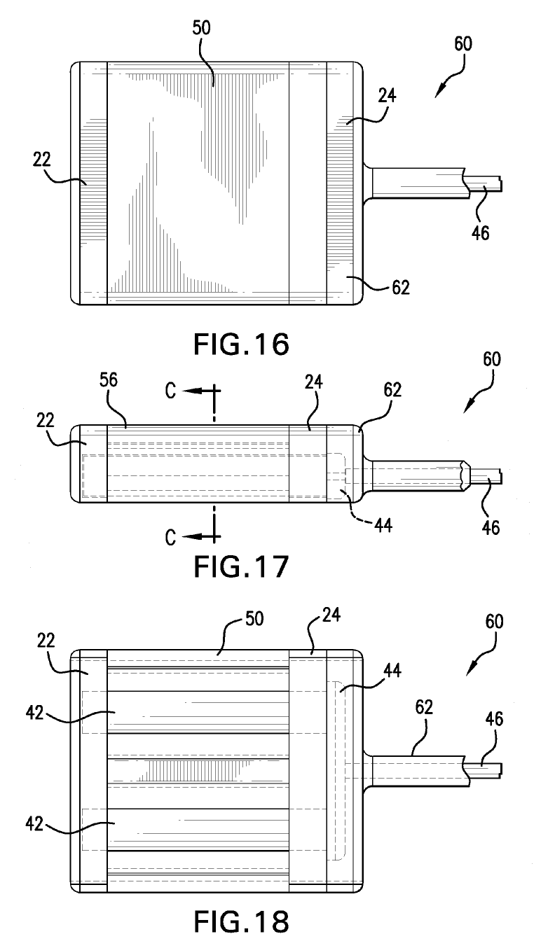

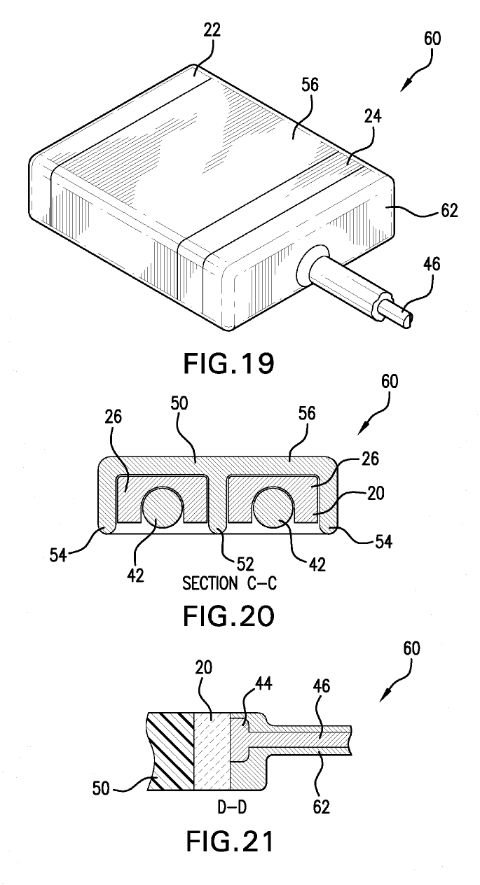

FIGS. 11 through 15 depict a floating electrode for an electrosurgical instrument of the present invention that is particularly suited to thermally treating patient tissue. As shown herein, the floating electrode 50 forms adjacent channels having a common flange 52, and lateral flanges 54 and wall 56. Flanges 52 and 54 have ends 58 formed to a radius. However, the present invention is not limited to the depicted design and includes alternate floating electrode embodiments, such as those described in U.S. Pat. Nos. 7,563,261 and 7,566,333 cited above, the contents of which are incorporated by reference herein in their entirety. Referring now to FIGS. 16 to 21, which depict a distal portion of an electrosurgical instrument of the present invention formed from the components of FIGS. 1 through 15, the distal portion of probe 60 is an assembly in which distal portions 42 of active electrode 40 are positioned within channel portions 34 of insulator 30. Floating electrode 50 is positioned between distal portion 22 and proximal portion 24 of insulator 20. Dielectric coating 62 covers flange 44 and conductor 46 of active electrode 40. Conductor 46 is connected by means within the probe 60 and electrical cable to a suitable electrosurgical generator.

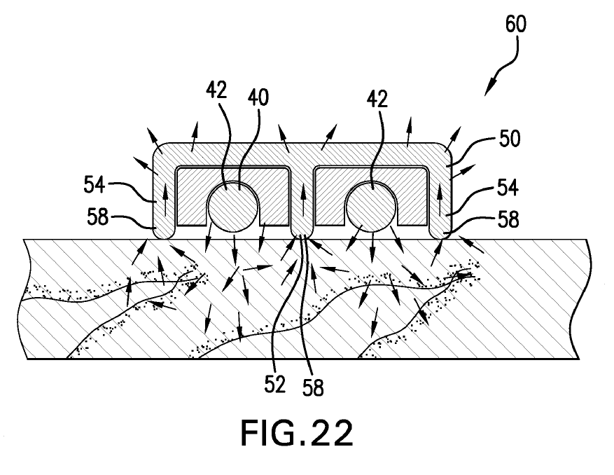

FIG. 22 depicts probe 60 in use, fully or partially submerged in irrigant (either endogenous to site or exogenously supplied). Flanges 52 and 54 of floating electrode 50 contact the tissue. Distal portions 42 of active electrode 40 may contact the tissue, or may contact the fluid in a gap between the electrode 40 and the tissue. Fluid surrounding the distal end of probe 60 is conductive. It may be supplied to the site as a conductive liquid such as standard saline, or may be supplied to the site as a non-conductive irrigant such as water, the fluid becoming conductive by contamination by body fluids such as blood, or by ablation by-products.

During use, current (indicated by arrows) flows from active electrode 40 to a return electrode (not shown), either at a remote site or mounted on the instrument 60. Current flows from distal portions 42 of active electrode 40 through tissue in contact with or in close proximity to portions 42. Some current flows through the tissue to the return electrode. A portion of the current flows through the tissue to radiused portions 58 of flanges 52 and 54 of floating electrode 50 in contact with the tissue to portions of floating electrode 50 in lower potential portions of the electric field. This current then flows from floating electrode 50 to conductive fluid in contact therewith, and then through the fluid to the return electrode. The efficiency of probe 60 for thermally treating tissue is enhanced by the elimination of regions of high current density. Such regions of high current density cause boiling of irrigant in close proximity, and arcing through the steam bubbles formed so as to vaporize tissue. The absence of these regions allows the device to be used at higher power levels for more rapid tissue treatment without creating these undesirable vaporizing sparks. Specifically, portions of flanges 52 and 54 which contact tissue are radiused so as to eliminate sharp corners which create regions of high current density. In addition, portions 42 of active electrode 40 are also rounded to eliminate sharp edges which create regions of high current density.

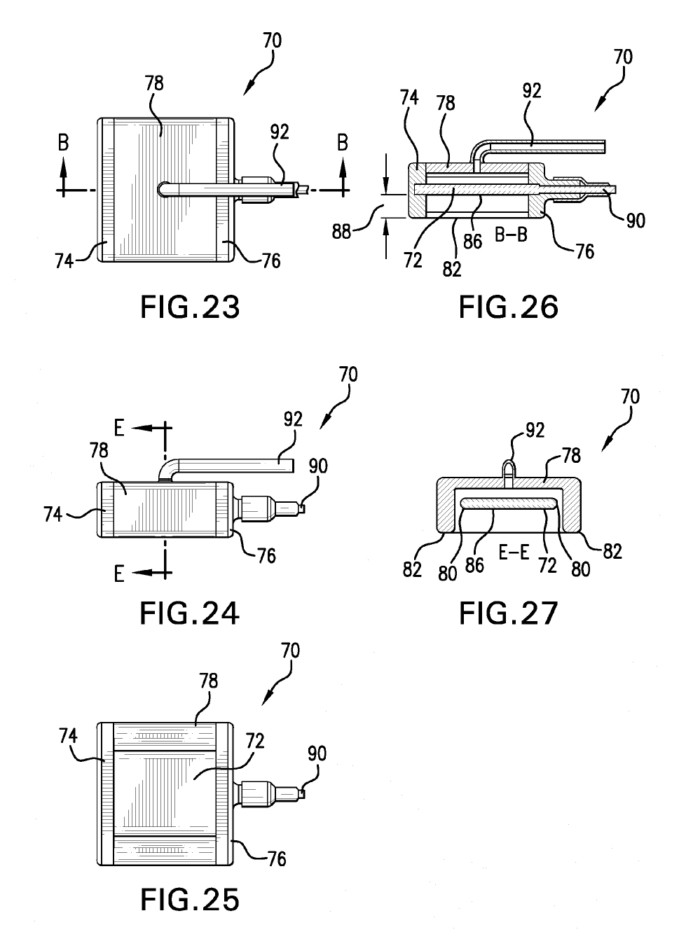

FIGS. 23 through 27 depict the distal portion of another thermal treatment electrosurgical instrument of the present invention. Probe 70 has a planar active electrode 72 suspended by distal dielectric end piece 74 and proximal dielectric end piece 76 in an inverted channel formed by floating electrode 78. Lateral edges 80 of active electrode 72, and edges 82 of floating electrode 78 are radiused. Lower surface 86 of active electrode 72 is recessed distance 88 from the plane of edges 82 of floating electrode 78. Conductor means 90 within probe 70 and cabling connect active electrode 72 to a suitable electrosurgical generator. Tubular member 92 is connected by means within probe 70 to an external conductive irrigant source.

Although the active electrode assembly is depicted as a having a square/rectangular profile and/or cross-section, the invention is not limited to the depicted configuration. So long as a particular configuration provides the requisite confined space, more particularly the presence of a fluid-fillable cavity defined between the active and floating electrodes, other geometries may be contemplated including, but not limited to, electrode assemblies having rounded, circular, elliptical, and polygonal profiles.

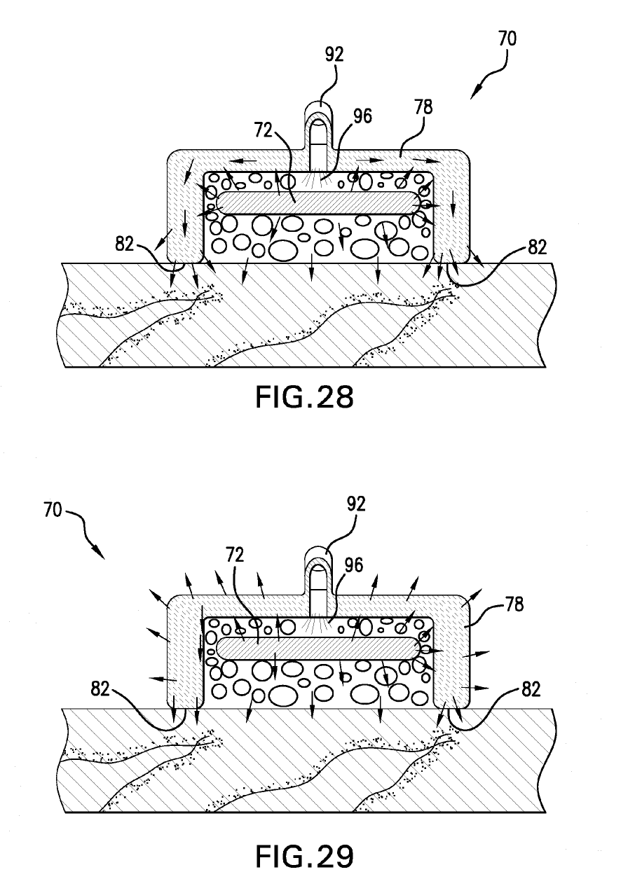

Referring now to FIG. 28, which depicts probe 70 in use in a “dry field”, conductive irrigant 96 may be supplied by tubular member 92 to the interior of the distal portion of probe 70, between the upper surface of active electrode 72 and the interior surface of floating electrode 78. Current (indicated by arrows) flows from active electrode 72 to a return electrode, either remotely located or on probe 70. A portion of the current flows through conductive liquid surrounding active electrode 72 to floating electrode 78 and therethrough to tissue in contact with or close proximity to edges 82. Current flowing from the floating electrode 78 to the tissue in this manner is conducted through direct contact or through conductive fluid in close proximity. A second portion of the current flows from the active electrode 72 to the tissue through conductive fluid between active electrode 72 and the tissue. Current flowing through conductive irrigant 96 heats irrigant 96 primarily in the regions in which active electrode 72 and floating electrode 78 are in close proximity. If the current flow is sufficiently high related to the flow rate of conductive irrigant 96, boiling of irrigant 96 occurs. Expanding steam and irrigant flow from tubular member 92 causes heated liquid and steam to flow into the region between active electrode 72 and the tissue. Thermal treatment of the tissue is accomplished through contact with heated liquid and steam, and through flow of current. The relative proportion of the two depends on the power supplied and the flow rate of conductive irrigant 96.

FIG. 29 depicts probe 70 in use in a conductive liquid environment. Conductive irrigant 96 is supplied by tubular member 92 to the interior of the distal portion of probe 70, between the upper surface of active electrode 72 and the interior surface of floating electrode 78. Current flows from active electrode 72 to a return electrode, either remotely located or on probe 70. A portion of the current flows through conductive liquid surrounding active electrode 72 to floating electrode 78 and therethrough to conductive liquid in contact with the exterior surfaces of floating electrode 78. A second portion of the current flows from the active electrode 72 to the tissue through conductive fluid between active electrode 72 and the tissue. Current flowing through conductive irrigant 96 heats irrigant 96 primarily in the regions in which active electrode 72 and floating electrode 78 are in close proximity. If the current flow is sufficiently high related to the flow rate of conductive irrigant 96, boiling of irrigant 96 occurs. Expanding steam and irrigant flow from tubular member 92 causes heated liquid and steam to flow into the region between active electrode 72 and the target tissue. Thermal treatment of the tissue is accomplished through contact with heated liquid and steam, and through flow of current. The relative proportion of the two depends on the power supplied and the flow rate of conductive irrigant 96. As with the instrument 60 previously herein described, probe 70 is designed to minimize or eliminate regions of high current density which cause arcing and tissue vaporization. Particularly, edges 82 of floating electrode 78 which contact target tissue are rounded to eliminate regions of high current density and arcing which may result therefrom. Also, lateral edges 80 of active electrode 72 are radiused to prevent arcing between active electrode 72 and the tissue or between electrode 72 and floating electrode 78. Lower surface 86 of active electrode 72 does not have features such as grooves, protuberances, recesses which increase current density, but is smooth. These features, individually and/or in combination, allow probe 70 to be used at higher power levels for more rapid tissue treatment without arcing and the resulting undesirable tissue vaporization.

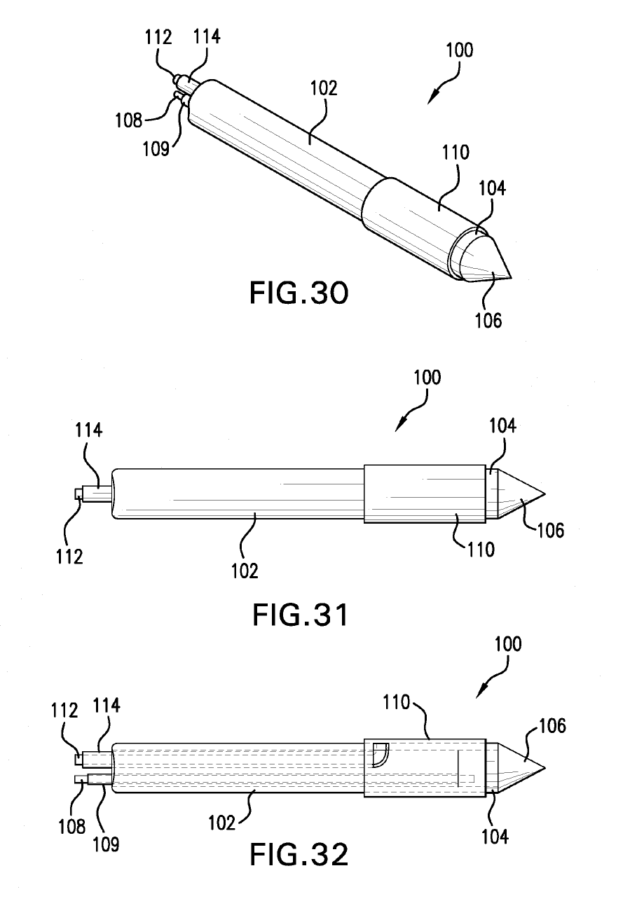

FIGS. 30 through 32 depict the distal portion of an electrosurgical instrument of the present invention that is particularly suited to the thermal treatment of tissue, more particularly sub-surface tissue treatment. While the previous embodiments are designed for surface treatment, electrosurgical instrument 100 thermally treats tissue into which it is inserted. Probe 100 is formed from a dielectric tube 102 having a sharpened, tapered or conical distal end 104 that facilitates atraumatic insertion into the target tissue. To distal end 104 is mounted active electrode 106 connected by conductor 108 insulated by dielectric coating 109 and means within probe 100 and cabling to a suitable electrosurgical generator. Tubular conductive member 110 is assembled to dielectric tube 102 near its distal end, and connected by conductor 112 insulated with dielectric coating 114 to a control element in the handle portion of probe 100. The control element has a first position in which conductor 112 is not connected to the electrosurgical generator, and a second position in which the conductor 112 is connected to the generator output such that when the generator is activated RF voltage is applied to member 110.

During use, probe 100, while energized, is first inserted into the tissue, tubular member 110 functioning as a floating electrode, the switching means being in its first position. When probe 100 is inserted to the desired depth, switching means is put in its second position and RF energy is supplied to conductive member 110 so as to treat tissue in close proximity.

Techniques for thermally treating tissue with RF energy by inserting an elongate electrode into the living tissue are well known in the art. When RF energy is applied, the tissue is resistively heated. However, a limitation of this technique derives from the fact that the tissue adjacent to the electrode is excessively heated leading to the tissue being desiccated, or even burned and charred. The desiccated tissue acts as a thermal and electrical insulator, effectively preventing the transmission of energy further into the tissue. This fundamentally limits the size of the lesion that can be attained by a single elongate electrode. Therefore, using RF energy to create uniform lesions larger than 2 centimeters in diameter in soft tissues using a single electrode is very difficult.

These limitations may be overcome by preventing the desiccation of tissues adjacent to the elongate electrode. In embodiments of the present invention hereafter described, conductive irrigant supplied to the distal end portion of the device is heated by RF energy flowing between an active electrode positioned within the lumen of a tubular distal portion electrode configured for insertion into tissue to be thermally treated. Heated fluid and/or steam flows from perforations in the tubular electrode into the tissue-electrode interface so as to thermally treat the tissue and prevent desiccation by the RF energy flowing through the tissue. In some embodiments the tubular outer electrode is not connected to the generator but rather is at a floating potential. Current flows from the active electrode, through the conductive irrigant to the floating potential electrode, and thereafter from the floating electrode through the tissue to a remotely located return electrode. In the case of these monopolar devices, tissue adjacent to the electrode is heated by RF energy passing from the floating electrode to the tissue and by the heated liquid and/or steam flowing from perforations in the floating electrode. In other embodiments, the tubular distal electrode is connected to the generator so that current flows from the active electrode through the conductive irrigant to the tubular return electrode and therefrom to the generator. As in the monopolar devices, the conductive irrigant is heated and heated liquid and/or steam passes through perforations in the outer return electrode into the electrode-tissue interface thereby heating the tissue. In the case of these bipolar devices, the surrounding tissue is heated by the liquid/steam only since the RF energy does not pass through the tissue. Desiccation of tissue adjacent to the electrode is prevented by the heated irrigant.

Monopolar devices of this type thermally treat tissue using a hybrid approach in which steam and heated fluid passing from perforations in a floating potential element hydrate tissue in proximity to the element so as to not only prevent desiccation, but to enhance the tissue's thermal and electrical conductivity; additionally this flow maintains the temperature in tissue adjacent to the element below that at which char is formed while supplying heat for thermal spread; The enhanced conductivity of the tissue allows the RF energy to flow, unimpeded by the desiccation that would present when using a conventional elongate RF electrode. Tissue in close proximity to the electrode is heated by a combination of energy supplied thereto by the heated fluid/steam and by resistive heating by the RF energy passing through the tissue. The rate of resistive heating by the RF energy at a location is proportional to the energy density at that location. The energy density decreases as the square of the distance from the electrode so that the portion of the heating attributable to the RF energy decreases rapidly with increasing distance from the electrode. At distances beyond a few millimeters from the electrode, heating of the tissue is predominantly by thermal conduction thereto of heat supplied to the site by the heated irrigant and steam formed by boiling of the irrigant within the floating electrode. The size of a lesion formed by a device of this type is determined by the level of power supplied to the device and the time that the power is supplied. Because the tissue into which the electrode is inserted is not desiccated during treatment, the treatment time is not limited thereby. By using the hybrid method of the present invention, a single small diameter electrode is able to thermally treat volumes of tissue greater than those producible by other single-electrode methods. Using a single 1.4 millimeter diameter monopolar electrode as described, the inventors are able to produce lesions (thermally treated regions) as large as 40 millimeters in diameter in 3 minutes

Bipolar devices (those in which the distal electrode serves as the return) thermally treat tissue using energy from the heated saline and steam formed therefrom only, the RF energy not flowing through the tissue. As with the monopolar device, the size of the lesion formed is determined by the level of the RF energy applied and the time for which it is applied.

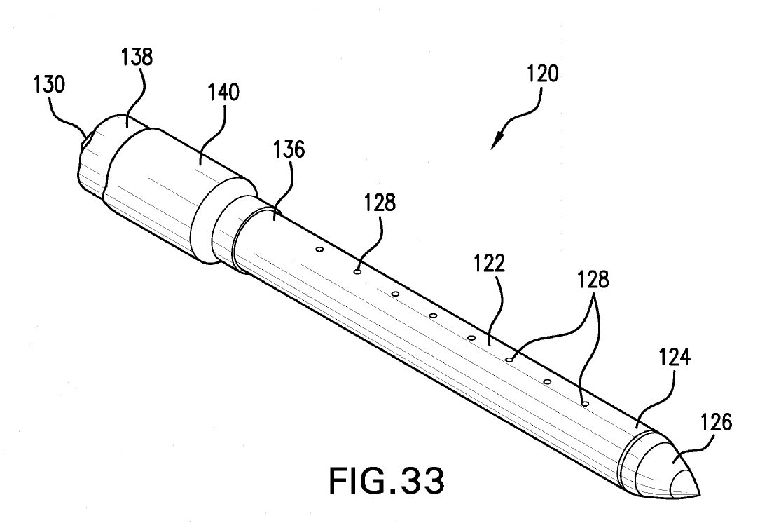

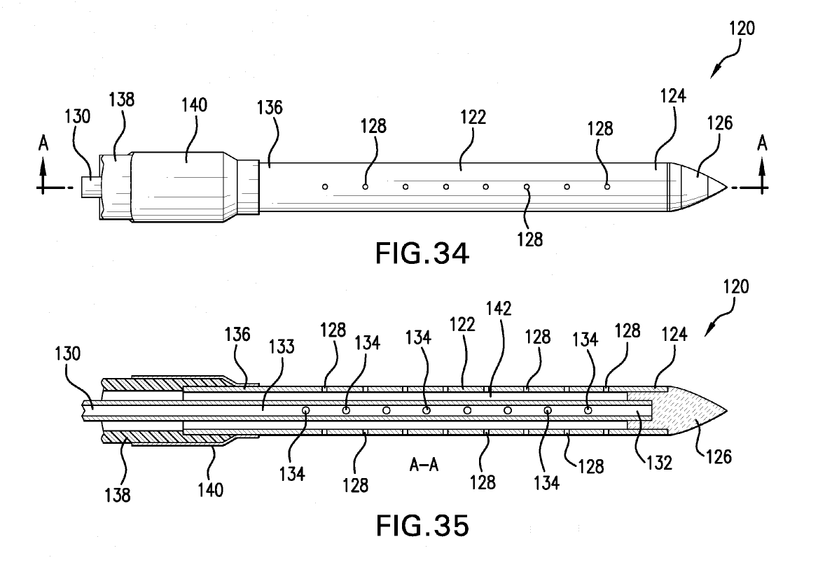

FIGS. 33 through 35 depict a monopolar thermal treatment device of the type previously described, —that is, one that treats tissue using a hybrid method incorporating steam and/or heated irrigant and RF energy. Probe 120, the distal portion of which is shown has a first conductive tubular member 122 having a distal end 124 in which is mounted dielectric member 126. Member 122 has a plurality of ports or perforations 128. Second tubular member 130 is coaxially positioned within member 122, and has a distal end 132 positioned within a recess in dielectric member 126. Second tubular member 130 with lumen 133 has a plurality of perforations 134. Proximal end 136 of member 122 is mounted to tubular member 138, the distal end of first tubular member 138 and proximal portion of member 122 are covered by dielectric coating 140. Second tubular member 130 is connected by means within probe 120 and cabling to a suitable electrosurgical generator. Region 142 is defined by the interior surface of first tubular member 122 and the exterior surface of second tubular member 130. Second tubular member 130 is connected by means within probe 120 and tubing to an external source for conductive irrigant.

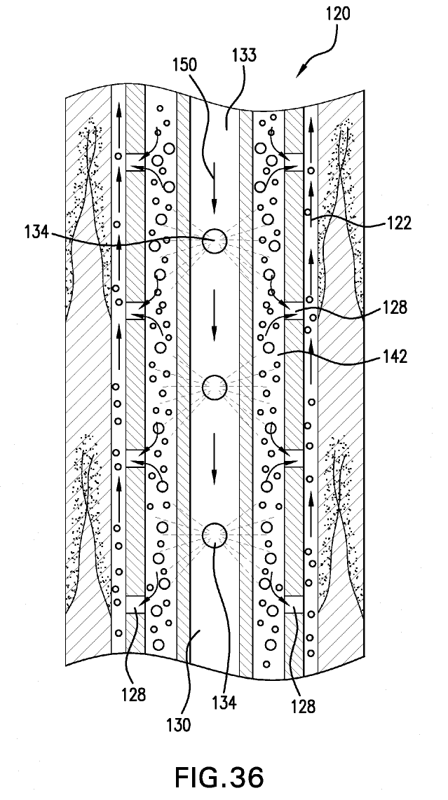

Referring now to FIG. 36 depicting a portion of probe 120 during use, probe 120 is inserted into tissue to be thermally treated. Conductive irrigant 150 is supplied to lumen 133 of second tubular member 130. Lumen 133, perforations 134, region 142, and perforations 128 together form a flow path for irrigant 150 from lumen 133 of second tubular member 130 to the region between the external surface of probe 120 and the tissue into which it is inserted. Current flows from second tubular member 132 which acts as an active electrode, through conductive irrigant 150 to first tubular member 122, and therethrough to adjacent tissue via conductive irrigant in the gap between probe 120 and the tissue, and finally to a return electrode (not shown), either remotely located or on probe 120. First tubular member 122 is not connected to the electrosurgical unit, but has a floating potential between that of the active electrode (second tubular member 130) and the tissue. Current flowing through irrigant 150 in region 142 heats the irrigant causing it to boil. Irrigant 150 flowing from region 142 through perforations 128 is a two-phase mixture of steam and liquid that heats tissue with which it is in contact. The relative portions of steam and liquid are determined by the flow rate of irrigant 150, and by the applied power level. Decreasing the power or increasing the flow rate will cause the liquid phase to increase. Tissue thermally treated by probe 120 is heated by the irrigant and by resistive heating caused by current flow. The RF energy supplied to probe 120 has characteristics selected to minimize arcing within bubbles in the irrigant, and between member 122 and adjacent tissue.

First tubular member 122 of device 100 has a uniform diameter throughout its length with heating of the irrigant occurring throughout that length in region 142. In other embodiments, the distal tubular member has a non-uniform cross-section. These embodiments have a proximal portion with a first diameter in which irrigant is heated, and a perforated distal portion of a second reduced diameter that is inserted into tissue, steam and heated irrigant flowing from the proximal heating/steam generating portion to the distal portions and therefrom into the tissue/device interface.

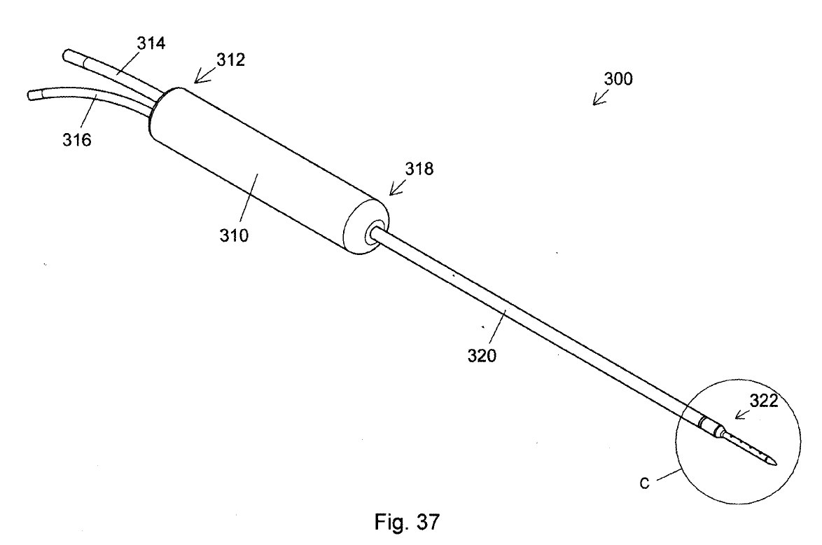

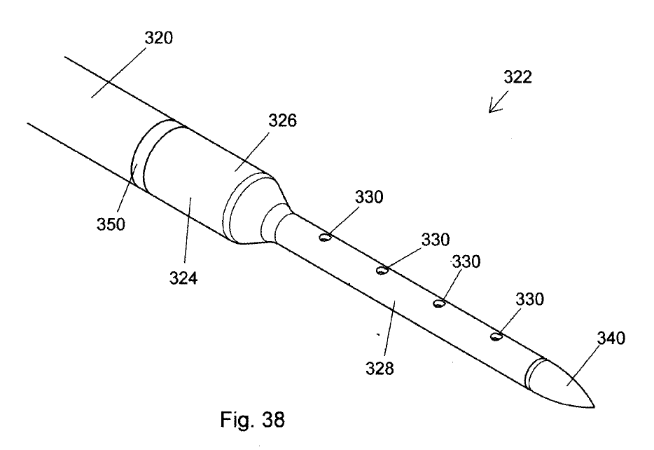

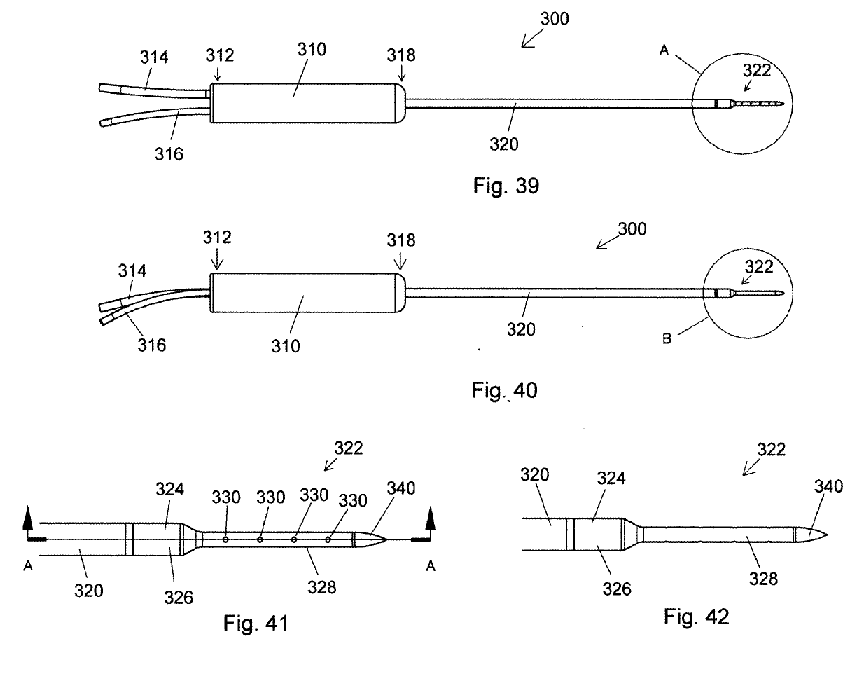

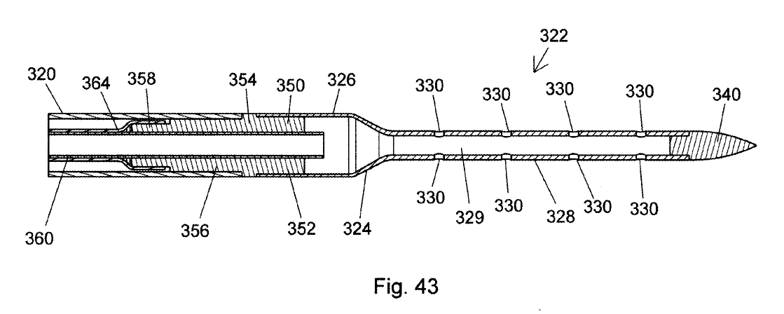

Device 300 subsequently described has a tubular distal element with a proximal “steam generator” portion configured for irrigant heating/steam generation and a distal portion configured for insertion into tissue. Referring now to FIGS. 37 through 43 depicting device 300 of the present invention, device 300 has a proximal handle portion 310 with a proximal end 312 from which pass tubular element 314 and power cable 316, and a distal end 318 from which protrudes elongate tubular element 320 with electrode assembly 322 at its distal end. Referring now to FIG. 38, electrode assembly 322 has a tubular distal element 324 with a proximal portion 326 configured for mounting to distal portion 352 of tubular insulator 350, and an elongate distal portion 328 to which is mounted sharpened element 340. Elongate distal portion 328 of tubular distal element 324 has a reduced diameter compared to proximal portion 326 and has perforations 330 formed along its length. Insulator 350 has a distal portion 352 configured for the mounting thereto of tubular distal element 324 as depicted, a flange 354 proximally adjacent, an elongate portion 356 proximal to flange 354, and a proximal-most portion 358. Portion 356 is configured for mounting to the distal end of tubular element 320. Tubular element 360 with lumen 361 has a distal end 362 which protrudes beyond the distal end of insulator 350. Tubular element 360 extends proximally to proximal handle portion 310 where it is connected by cable 316 to a suitable electrosurgical generator (not shown), and by tubular element 114 to a suitable conductive irrigant source (not shown). Polymeric sheath 364 surrounds tubular element 360 and overlaps proximal-most portion 358 of insulator 350. Tubular distal element 324 is electrically isolated from tubular element 360 and tubular element 320. Tubular elements 320, 324 and 360 are formed from a suitable metallic material such as, for instance, stainless steel. Insulator 350 is formed from a suitable ceramic material such as, for instance, alumina or zirconia, or from a suitable polymeric material such as, for instance, PEEK. In some embodiments sharpened distal element 340 is made from a metallic material, in other embodiments it is made from a suitable dielectric material.

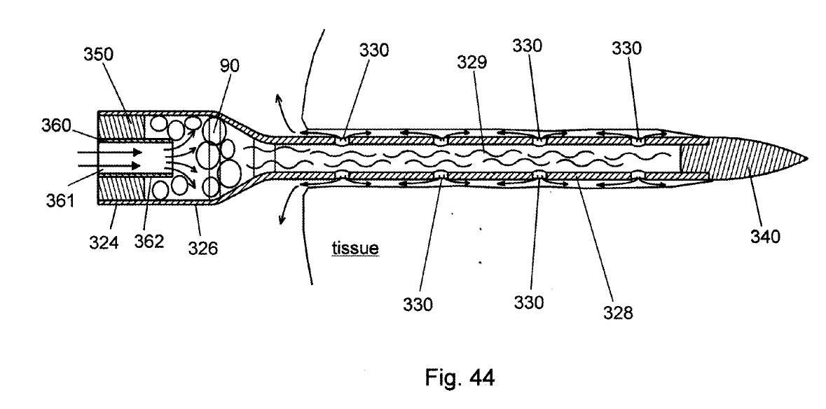

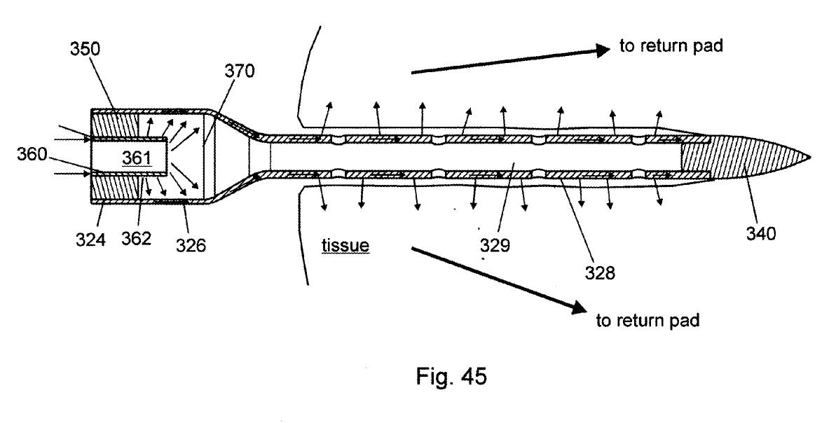

FIG. 44 depicts the flow of irrigant and steam formed therefrom during use when distal portion 328 of tubular distal element 324 is inserted into tissue for thermal treatment of the tissue. FIG. 45 depicts the flow of RF energy during the thermal treatment. In use, tubular member 360 functions as an active electrode. Irrigant supplied via lumen 361 of conductive tubular member 360 enters proximal portion 326 of tubular distal element 324. As seen in FIG. 45, radio frequency energy flows from tubular member 360 through the conductive irrigant to proximal portion 326 of distal tubular member 324, then through tubular member 324 to distal portion 328 of tubular member 324, from which it flows to tissue adjacent to distal portion 328 of tubular member 324. This flow may be through tissue in contact with distal portion 328 or conductive irrigant present in a gap between distal portion 328 of tubular member 324 and the tissue. Current flowing through irrigant between tubular member 360 and proximal portion 326 of tubular member 324 causes the irrigant to boil forming steam that flows to distal portion 328 of tubular member 324 and through lumen 329 of distal portion 328 to perforations 330. Thereafter steam exiting perforations 330 transfers heat to tissue adjacent to distal portion 328 of tubular member 324. Steam and irrigant flow proximally in the gap between distal portion 328 of tubular element 324 and the adjacent tissue and thereby exit the site. Tissue is heated by the flow of RF energy from distal portion 328 of tubular element 324 to the tissue, and by steam flow from perforations 330. The heating effect of the RF energy decreases rapidly with increasing distance from distal portion 328 of tubular element 324. Flow of steam from perforations 330 ensure that tissue adjacent to distal portion 328 is not desiccated thereby preventing an associated increase in resistance and a resulting decrease in the flow of RF energy. The flow of steam from perforations 330 is maintained until a predetermined region of the tissue is heated to temperatures sufficient to necroses the tissue.

While device 300 may be used to thermally treat tissue in a mode in which heated irrigant only flows from perforations 330, in another mode in which a two-phase flow of heated irrigant and steam flows from perforations 330, and a third mode in which steam only flows from perforations 330. In a preferred method, the third mode is used; steam flowing from perforations 330 thermally treats the tissue, a portion of the steam condensing to liquid after exiting perforations 330 so as to create phase-change heating of adjacent tissue. The balance of the steam exits proximally in a gap formed between the outer surface of distal portion 328 of tubular element 324 and adjacent tissue. The volume of heated liquid exiting via this gap when device 300 is operated in this steam-only mode is much less than if device 300 were used in a mode in which primarily heated irrigant liquid were used. This is advantageous since heated liquid flowing from the gap may contact adjacent tissue and organs causing unintended thermal injury thereto.

Additionally, when operating in a steam only mode, RF energy flows through device 300 to tissue only when sufficient irrigant is present in the steam generator (proximal) portion 326 of tubular distal element 324 to bridge the gap between the distal end of active electrode 360 and proximal portion 326 of tubular element 324. Current flow through the irrigant boils the irrigant, the steam flowing to distal portion 328 of tubular element 324. When irrigant bridges the gap between active electrode 360 and proximal portion 326 of tubular element 324 within the steam generator portion of tubular member 324, the impedance of the conductive path so formed matches that of the electrosurgical generator and RF current flows from the generator. When the region is filled with steam rather than conductive irrigant, the impedance of the path is high and RF current does not flow from the generator, the production of additional steam is prevented, and the flow of additional energy to the region is prevented. Device 300 continues in this mode until additional irrigant is supplied to the steam generator region 326 of tubular element 324. In this manner, RF energy is only supplied to the site when steam is being generated. It is not possible for RF energy to be supplied in the absence of conductive irrigant, the flow of energy being regulated by the irrigant flow so long as the flow rate does not exceed the amount that can be vaporized by power supplied by the generator. The power setting of the generator, then, determines the maximum irrigant flow rate that can be used when treatment is to be in a steam-only mode.

Arcing in the steam-generator (proximal) portion 366 of tubular element 364 is undesirable and may lead to destruction of assembly 322. Additionally, such arcing would allow energy to be supplied to the site in the absence of irrigant or steam, also an undesirable condition. Accordingly, it is necessary that the RF energy supplied to device 300 have a maximum voltage at high impedance that is insufficient to cause arcing between active electrode 360 and tubular element 324, and insufficient to cause arcing within bubbles formed in the proximal steam generator (proximal portion) 326 of tubular element 324. For devices 300 configured for creating lesions up to about 40 millimeters, it is desirable to limit the voltage supplied to device 300 to about 300 Volts or less at high impedances. For smaller (miniature) devices configured to create smaller lesions it may be necessary to limit the voltage supplied to probe 300 to 100 Volts or less at high impedances. A generator configured for use with device 300 should have a maximum output voltage of 100 to 300 Volts depending on the size and intended use of the device 300. Device 300, when the steam generator portion 326 of tubular element 324 is partially or completely filled with conductive irrigant has an impedance generally between 20 and 80 Ohms. Accordingly, it is necessary that the RF generator have an output impedance of about 20 Ohms.

To summarize, an RF generator configured for use with device 300 will have an output in the radio frequency range, preferably a few hundred kilohertz. The output voltage at high impedance (open circuit) will be 100 to 300 Volts, and the output impedance will typically be in the 20 to 40-ohm range. The maximum power output will be determined by the size of lesions to be created, but will generally be between 100 and 200 Watts.

While the general purpose electrosurgical generators present in operating rooms have suitable frequencies and power output, their output voltage is generally above 1,000 Volts. They are designed to deliver power to devices having an impedance between 200 and 600 ohms. Accordingly, generators of this type do not have output characteristics that allow their use with device 300.

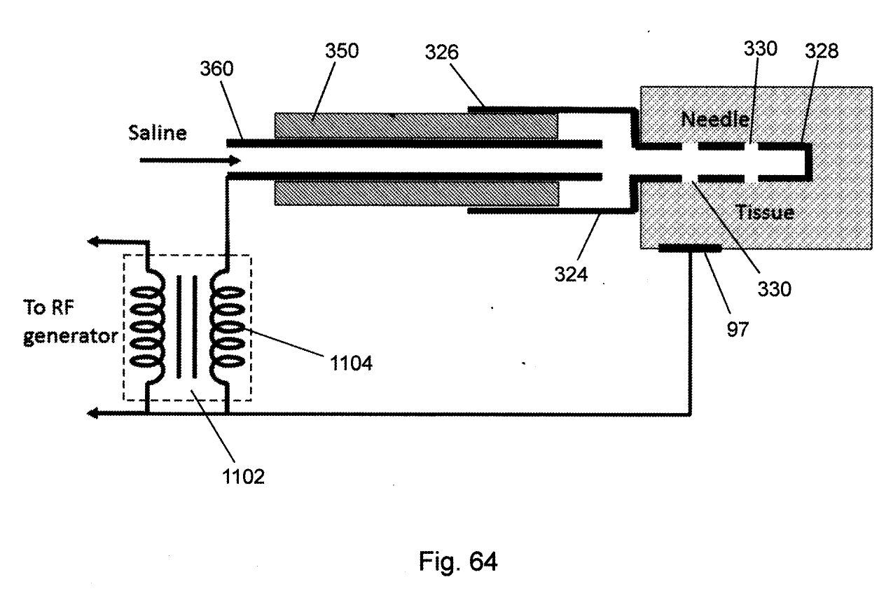

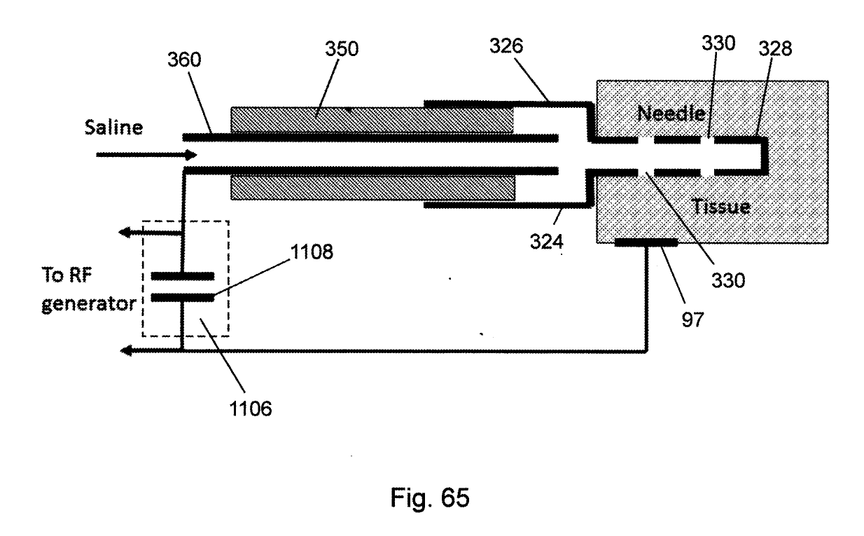

The output of these general-purpose generators may be modified by suitable adaptor circuitry to match the required characteristics listed above. A first such adaptor circuit 1102 is depicted in FIG. 64. Adaptor circuit 1102 comprises a step-down transformer 1104 that both decreases the maximum voltage applied to device 300 and increases the effective load of the device 300. Adaptor circuit 1102 matches the impedance of the general-purpose generator to that of device 300. The use of adaptor circuit 1102 does not decrease the system efficiency since transformer 1104 is a passive component. The primary winding of step-down transformer 1104 is electrically connected between the monopolar output of the general-purpose generator and the return electrode socket of that generator. The secondary winding of transformer 1104 is connected between the return electrode socket (and remotely located return electrode 97 electrically connected thereto) and the input cable 316 (see FIG. 37) of device 300

A second adaptor circuit 1106 is depicted in FIG. 65. Adaptor circuit 1106 comprises capacitor 1108 connected in parallel with device 300. Connecting capacitor 1108 in this manner lowers the maximum voltage applied to device 300 as required to prevent arcing within proximal steam generator portion 326 of tubular element 324 of device 300. The output current of standard multipurpose generators is limited. Placing a capacitive load with sufficiently low impedance will restrict the output voltage of the generator well below the generator's maximum output. Because capacitor 1108 does not dissipate energy and does not load the generator (no resistive energy dissipation), the efficiency of the system is not decreased since capacitor 1108 is a reactive load. The resistive load supplied by device 300 uses the entire output power of the generator to create heated irrigant and steam with capacitor 1108 consuming the excessive current and restricting the voltage.

Adaptor circuits 1102 and 1106 are presented as illustrative examples only. Other adaptor circuits may be constructed that restrict the maximum voltage of a general purpose electrosurgical generator, and that match the output impedance of the generator to that of device 300. Such adaptors may contain a combination of capacitors, inductors and transformers as well as resistive components. These adapters may also include active networks, and or feedback networks in order to form the desired volt-ampere characteristics of the electrical energy source. So long as the adaptor circuitry is connected to the monopolar output and return receptacle of a general-purpose generator with an output connected to device 300 and to the return electrode (on the device or remotely located on the patient) the system so formed falls within the scope of this invention.

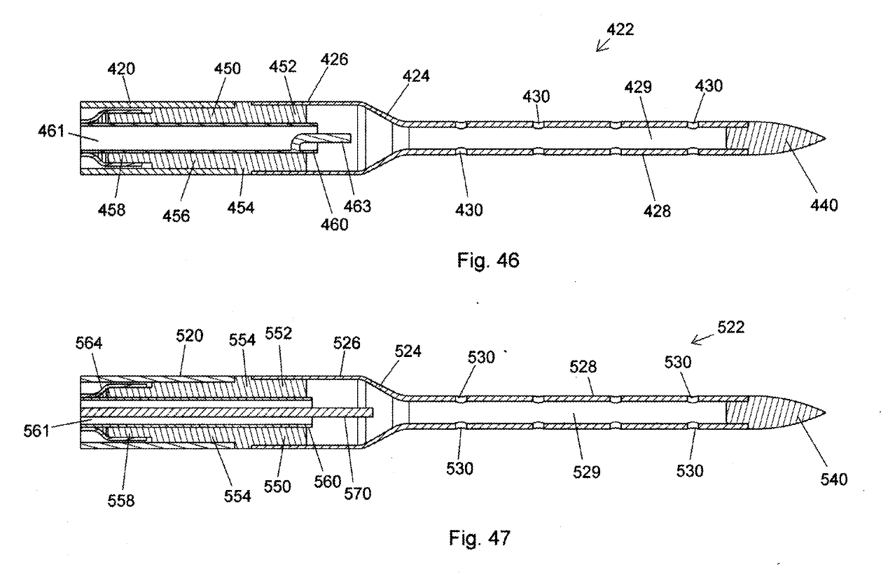

The configuration of distal assembly 322 may be modified without departing from the principles of the present invention. In FIG. 46, distal assembly 422 of probe 400 of the present invention is alike in all aspects of form and function to distal assembly 322 of probe 300 except as specifically subsequently described. Conductive tubular member 460 has positioned at the distal end of lumen 461 conductive distal element 463, the purpose of element 463 to assist in the boiling of irrigant in the proximal portion 426 of tubular element 424. Element 463 provides an enhanced conductive path for RF energy to flow from tubular element 460 through irrigant exiting the distal end of lumen 461 to proximal portion 426 of tubular element 424 as previously described.

An alternate embodiment 500 of the present invention, the distal assembly 522 of which is depicted in FIG. 47, is alike in all aspects of form and function to distal assembly 322 of probe 300 except as specifically subsequently described. Tubular member 560 is formed of a dielectric material. Elongate metallic element 570, positioned within lumen 561 of tubular element 560, functions as an active electrode such that irrigant flowing from the distal end of lumen 561 contacts element 570 and proximal portion 526 of tubular element 524 providing a conductive path for RF energy. As in previous embodiments, this RF energy boils the irrigant creating steam for the thermal treatment of tissue.

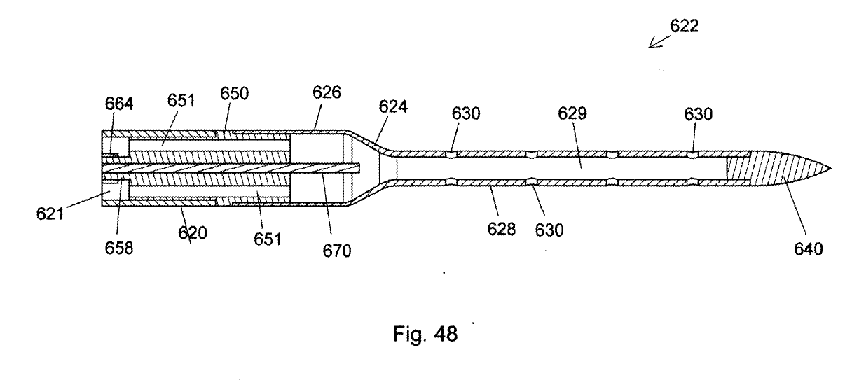

Alternate embodiment thermal treatment device 600, the distal assembly 622 of which is depicted in FIG. 48, is like device 500 in all aspects of form and function except as specifically subsequently described. Tubular element 560 is eliminated. Irrigant flows to the distal end of device 600 through lumen 621 of elongate tubular element 620 and therefrom through lumens 651 of insulator 650. Elongate metallic element 670, functions as an active electrode such that irrigant flowing from the distal ends of lumen 651 contacts element 670 and proximal portion 626 of tubular element 624 providing a conductive path for RF energy. As in previous embodiments, this RF energy boils the irrigant creating steam for the thermal treatment of tissue.

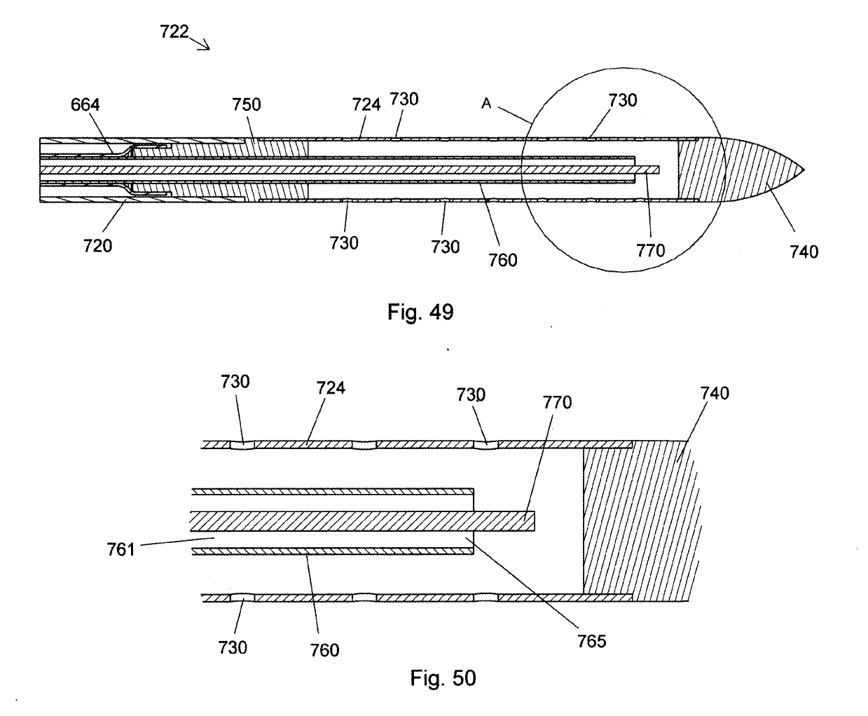

In other embodiments of the present invention, irrigant is boiled to steam at the distal end of the distal assembly, or along the length of the assembly. FIG. 49 depicts the distal portion 722 of a thermal treatment device 700 of the present invention alike in form and function to device 500 (FIG. 47) in all aspects of form and function except as specifically subsequently described. Distal tubular element 724 has a constant diameter equal to that of proximal portion 726 mounted to insulator 750. Tubular member 760, formed from a suitable dielectric material, has its distal end in proximity to distal element 740. Elongate metallic element 770 positioned within lumen 761 of tubular element 760 extends beyond the distal end of element 760. Elongate metallic element 770 functions as an active electrode such that irrigant flowing from the distal end of lumen 761 contacts element 770 and the distal portion of tubular element 524 providing a conductive path for RF energy. As in previous embodiments, this RF energy boils the irrigant creating steam near the distal end of tubular element 724, the steam flowing proximally to perforations 730 in tubular element 724, and therethrough to tissue in proximity to tubular element 724.

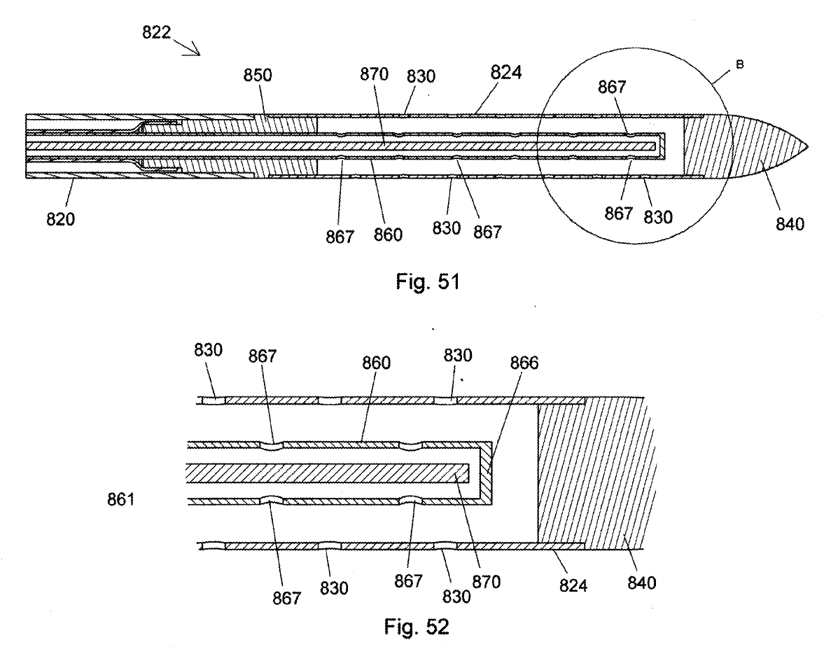

Like device 700 depicted in FIGS. 49 and 50, alternate embodiment device 800, the distal portion 822 of that is depicted in FIGS. 51 and 52, has a distal tubular portion 824 of constant diameter. Indeed, device 800 is alike in form and function to device 700 in all aspects of form and function except as specifically hereafter described. Tubular member 860 formed of a suitable dielectric material has a closed distal end 866. Elongate metallic element 870, positioned within lumen 861 of tubular element 860 ends proximal to closed distal end 866 so that element 870 is encapsulated within tubular element 860 except for perforations 867 formed in tubular element 860. Elongate metallic element 870 functions as an active electrode. Irrigant supplied to tubular element 860 flows through perforations 867 to the lumen of tubular element 824 thereby creating a path for RF energy to flow from elongate element 870 to tubular element 824 thereby boiling irrigant to create steam. This steam exits tubular member 824 via perforations 830 formed therein to thermally affect tissue in proximity as previously herein described.

Embodiments of the present invention previously herein described have a cylindrical tubular geometry configured for producing thermal effects that propagate radially outward from the device in tissue into which the device is inserted. In other embodiments hereafter described, the geometry of the distal portion of the tubular distal element has planar faces with an array of perforations formed thereon. In some embodiments intended for insertion into tissue, these perforations are formed in both planar surfaces, in others intended for treating surfaces and tissue immediately therebelow, a single planar surface has perforations.

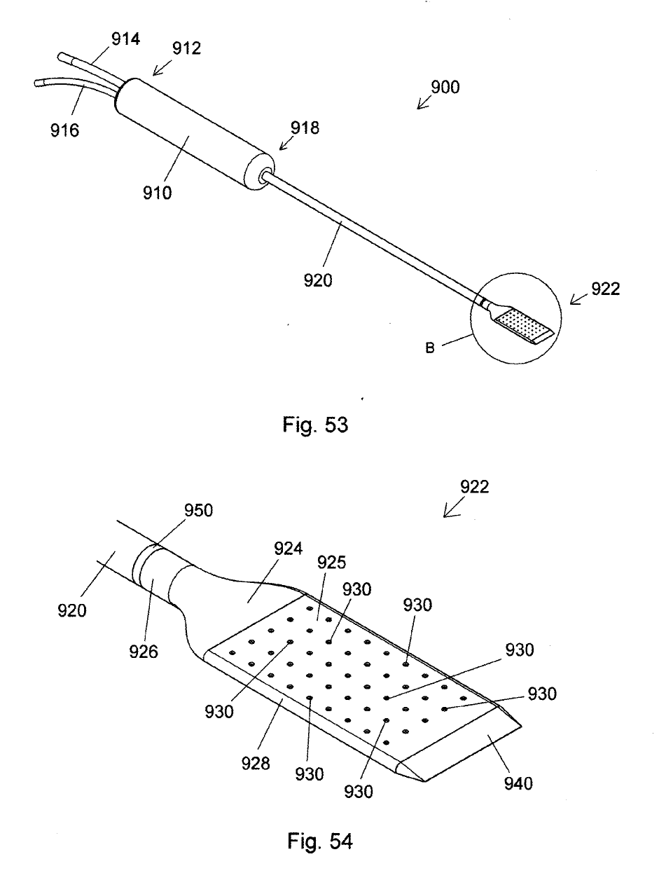

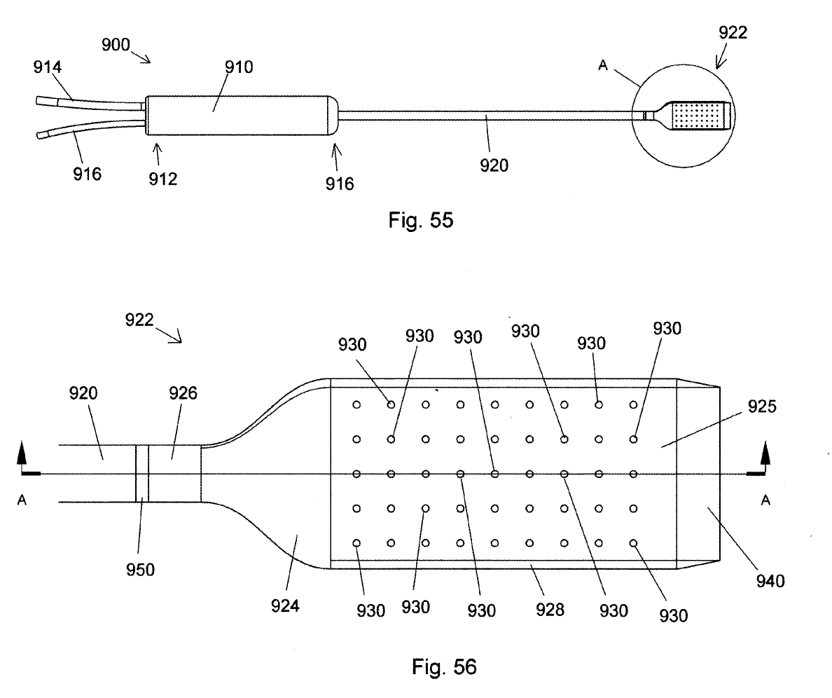

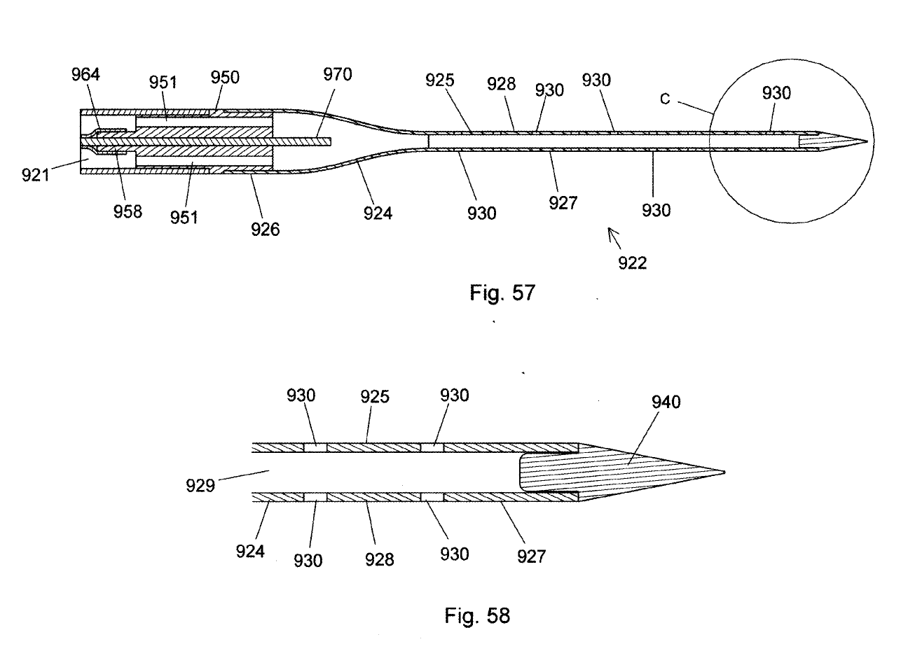

FIGS. 53 through 58 depict thermal treatment device 900 that is identical in all aspects of form and function to device 600 (FIG. 48) except as subsequently herein described. Distal portion 928 of distal tubular element 924 has formed thereon first and second parallel planar surfaces 927 and 929 respectively. Surfaces 927 and 929 have formed in them a plurality of perforations 930. The flow and boiling of irrigant to form steam for the thermal treatment of tissue is accomplished in the same manner as with device 600 except that rather than supplying steam to treat adjacent tissue via a linear array of perforations 630 (FIG. 48), thermal treatment device 900 supplies steam to adjacent tissue via a two-dimensional array of perforations 930 in first and second planar surfaces 927 and 929 to tissue surfaces adjacent thereto.

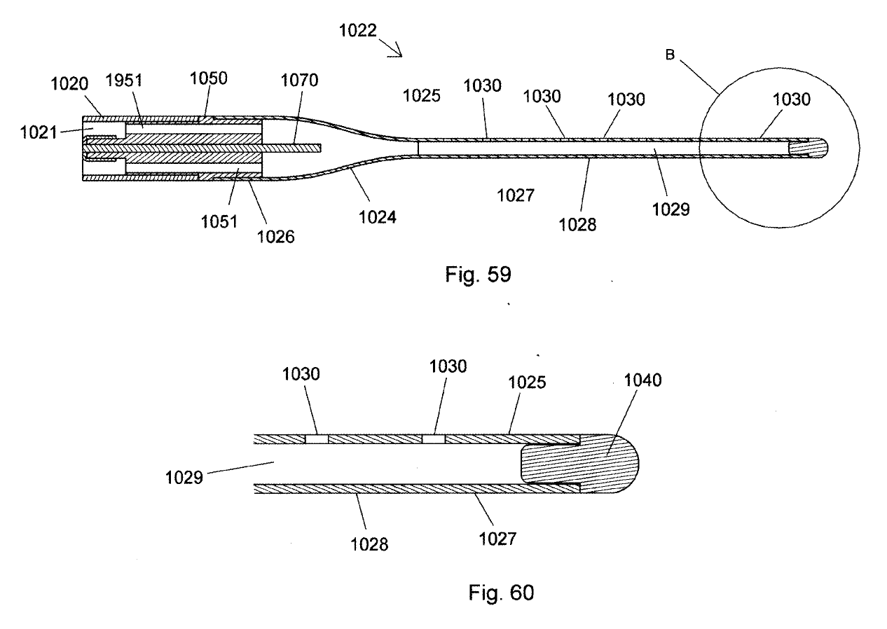

Alternate embodiment thermal treatment device 1000, depicted in FIGS. 59 and 60 is identical in all aspects of form and function to device 900 except as specifically hereafter described. Perforations 1030 are formed in first planar surface 1027 only.

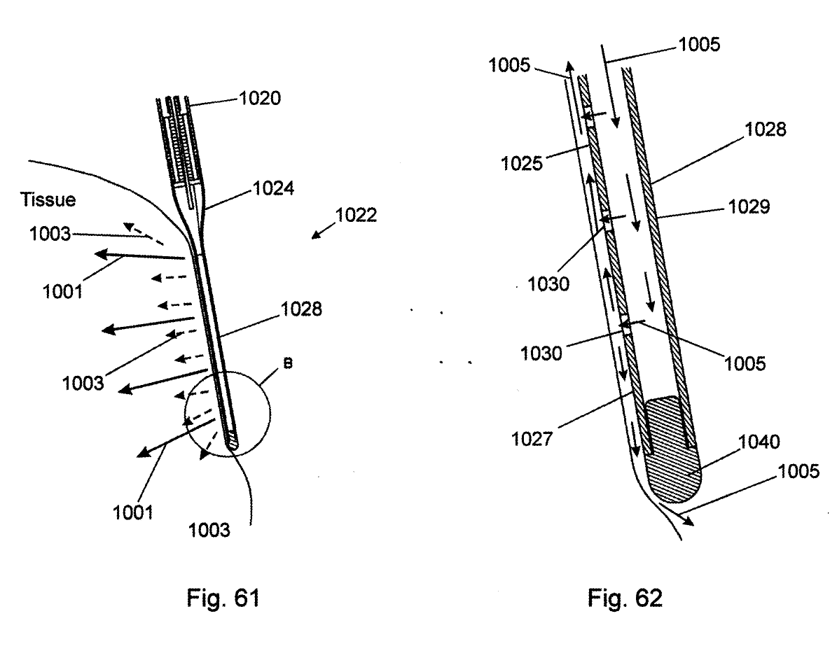

Sharpened distal element 940 is replaced by rounded distal element 1040. Thermal treatment device 1000 is configured for the thermal treatment of tissue at and in proximity to a surface. Device 1000 has geometry configured for producing thermal effects that propagate primarily normal to first planar surface 1027 to which first planar surface 1027 is applied. FIGS. 61 and 62 depict device 1000 thermally treating tissue, first planar surface 1027 of distal portion 1028 of distal element 1024 of assembly 1022 being positioned adjacent to a surface of the tissue. In FIG. 61 the dashed arrows 1003 indicate the flow of RF energy from distal element 1024 into the tissue, while solid arrows 1001 depict thermal energy. FIG. 62 depicts the flow 1005 of heated irrigant and/or steam distally within distal portion 1028 of distal element 1024 to perforations 1030 from which it flows into the interface between tissue and first surface 1027 of distal portion 1028, and exiting therefrom at the perimeter of distal portion 1028. Tissue adjacent to first surface 1027 of distal portion 1028 is hydrated by heated irrigant and/or steam exiting from perforations 1030 thereby enhancing the electrical conductivity of the tissue. The tissue is heated by the flow therethrough of RF energy flowing to a remotely located return electrode (not shown) and by the heated irrigant and/or steam.

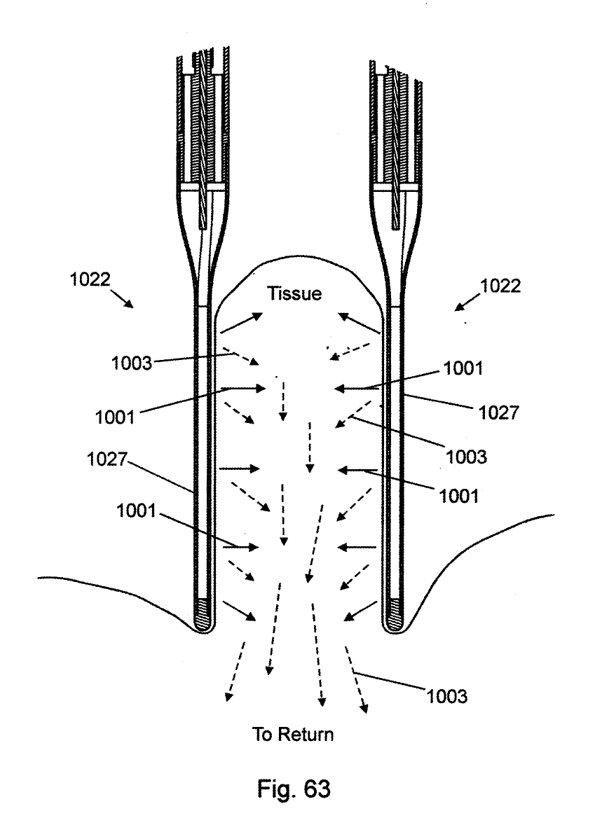

FIG. 63 depicts two probes 1022 symmetrically positioned on opposite sides of a tissue mass for the purpose of thermally treating the tissue. The flow of heated irrigant/steam is as previously described. Thermal energy 1001 flows into the tissue as depicted and RF energy 1003 flows through tissue to a remotely located return electrode (not shown).

Embodiments previously herein described are monopolar devices that have a tubular distal element that is not connected to the electrosurgical generator but rather has a floating potential. RF energy flows through the floating electrode on its way to a remotely located return electrode. Other embodiments of the present invention are configured to be bipolar, the distal tubular event being connected to the return receptacle of the electrosurgical generator so as to function as a return electrode.

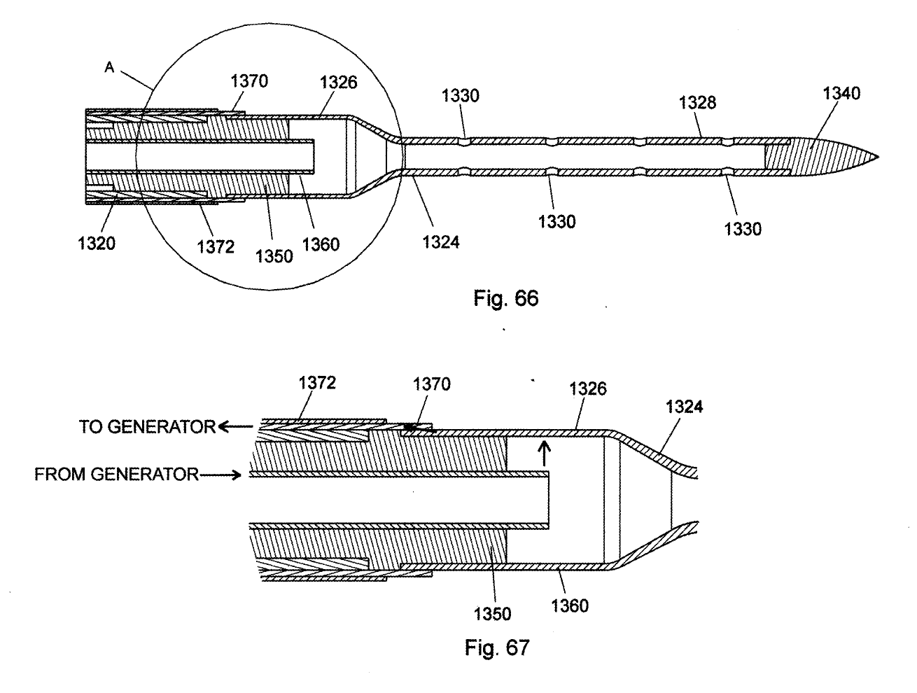

FIGS. 66 and 67 depict the distal portion of a bipolar thermal treatment device 1300 of the present invention. Device 1300 is like device 300 (FIGS. 37 through 43) in all aspects of form and function except as specifically subsequently described. Proximal portion 1326 of distal tubular element 1324 is connected via conductive tubular member 1370 to which it is mechanically and electrically attached and cabling to the return receptacle of an electrosurgical generator during use so that tubular element 1324 functions as a return electrode. During use, RF current flows from the electrosurgical generator to active electrode 1360, through conductive irrigant to proximal portion 1326 of distal tubular element 1324, and thereafter through conductive tubular member 1370 and via conductive means and cabling to the return receptacle of the electrosurgical generator. Proximal portion 1326 of tubular element 1324 coupled with the distal end of active electrode 1360 together function as a steam generator. RF energy flowing through the conductive irrigant heats the irrigant causing it to boil creating steam which flows to distal portion 1328 of tubular element 1324, exiting through perforations 1330 to thermally treat adjacent tissue. Unlike thermal treatment using monopolar device 300, when using bipolar device 1300 no RF energy flows through the tissue. Heating of the tissue is by heated irrigant and/or steam passing from perforations 1330 only.

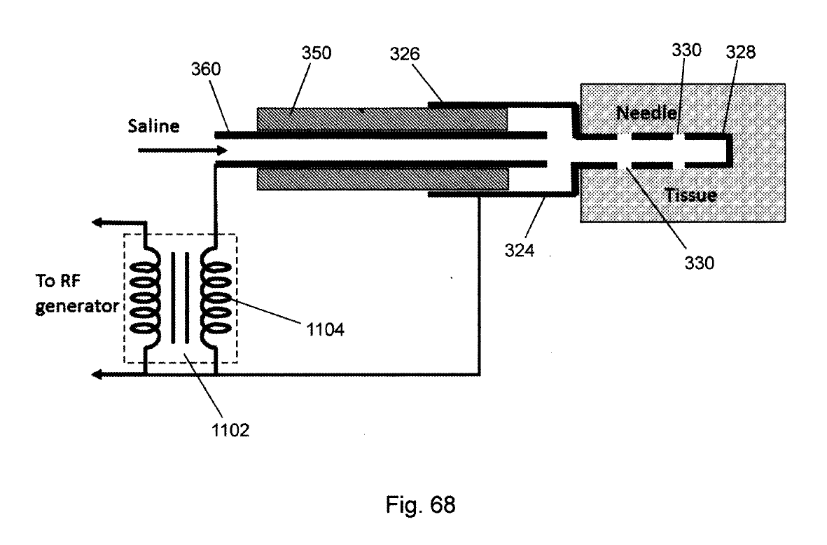

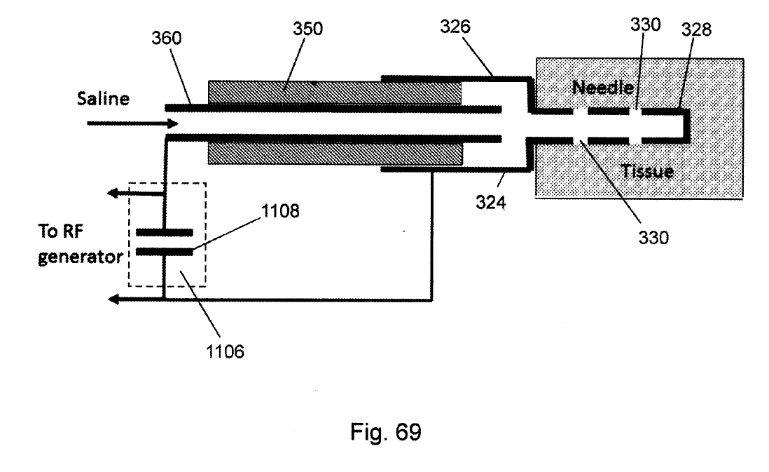

The output characteristic requirements for an electrosurgical generator for powering device 1300 are the same as those for device 300. Adaptor circuitry previously herein described for use with device 300 may be used for bipolar device 1300 as well. FIG. 68 schematically depicts previously described adaptor circuit 1102 comprising step-down transformer 1104 connected to device 1300. The primary winding of step-down transformer 1104 is electrically connected between the monopolar output of the general-purpose generator and the return electrode socket of that generator. The secondary winding of transformer 1104 is connected between the return electrode socket (and remotely located return electrode 97 electrically connected thereto) and the input cable 316 (see FIG. 37) of device 1300. FIG. 69 schematically depicts previously described adaptor circuit 1106 comprising 1108 connected to device 1300. When connected as depicted, adaptor circuit 1106 functions in the same manner as previously described. Rather than being connected to remote return electrode 97 as shown in FIG. 65, adaptor circuit 1106 is connected to tubular distal element 1324 which functions as a return electrode.

### INDUSTRIAL APPLICABILITY

The minimally invasive monopolar and bipolar electrosurgical instruments of the present invention find utility in the area of remote tissue ablation and lesion formation, to destroy tumors, form lesions, denaturize, desiccate, coagulate and ablate soft tissues, as well as to drill, cut, resect and vaporize soft tissues, with or without externally supplied conductive or non-conductive liquids (i.e., in the context of both wet and dry field electrosurgery). More particularly, the electrosurgical instruments of the present invention are designed to heat tissue from the outside in, to provide homogeneous energy deposition using less power, which in turn yields a highly homogeneous lesion.

In this manner, the electrosurgical instruments of the present invention allow one to effectively and efficiently control of the shape and size of the lesion formed, to thereby avoid unnecessary complications and undesired side effects. Such instruments are particularly useful in the context of oncological, ENT, urological, gynecological, and laparascopic procedures, as well as in the context of general surgery.

All patents and publications mentioned herein are incorporated by reference in their entirety. Nothing herein is to be construed as an admission that the invention is not entitled to antedate such disclosure by virtue of prior invention.

While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention.

Other advantages and features will become apparent from the claims filed hereafter, with the scope of such claims to be determined by their reasonable equivalents, as would be understood by those skilled in the art. Thus, the invention is intended to be defined not by the above description, but by the following claims and their equivalents.

What is claimed:

1. An electrosurgical instrument for thermal treatment of target tissue comprising:
(d) an elongate shaft having a proximal end, a distal end and a longitudinal axis, wherein said proximal end is configured for connection to a power source and said distal end is configured for connection to an electrode assembly;
(e) a means for supplying an irrigant to said electrode assembly;
(f) an electrode assembly mounted to the distal end of said elongate shaft, said electrode assembly comprising:
i. an active electrode formed from an electrically conductive material and having a proximal end connectable to said power source via one or more connecting elements disposed within said shaft and an exposed distal end configured to contact irrigant delivered to said electrode assembly by said irrigant supply means;
ii. an insulator formed from a nonconductive dielectric material and disposed about the periphery of a portion of said active electrode, wherein said tubular insulator includes at least one irrigation lumen in fluid communication said irrigant supply means, further wherein the exposed distal end of said active electrode is distal to a distal end of said tubular insulator;
iii. a floating electrode formed from an electrically conductive material that is in close proximity to said active electrode but electrically insulated therefrom by means of said tubular insulator, wherein said floating electrode is not directly electrically connected to either the elongate shaft or the power source, further wherein said floating electrode has a proximal portion disposed about the periphery of a distal portion of said insulator, a distal end that is distal to the exposed distal end of said active electrode, and an intermediate portion provided with one or more perforations along its length;
iv. a distal element mounted to the distal end of said floating electrode; and
v. an enclosed steam generating cavity in fluid communication with said at least one irrigation lumen that is (a) surrounded by at least said intermediate portion of said floating electrode and (b) bounded at its proximal end by said insulator and at its distal end by said distal element;
wherein:
powering said active electrode in the presence of said irrigant results in flow of current from said active electrode to said floating electrode, heating of the irrigant, and generation of heated irrigant and/or steam in at least said steam generating cavity, wherein flow of heated irrigant and/or steam flow from said steam generating cavity to said target tissue is controlled by said one or more perforations, further wherein said target tissue is thermally treated upon contact with said heated irrigant and/or steam.

2. The electrosurgical instrument of claim 1, wherein said insulator is tubular in shape.

3. The electrosurgical instrument of claim 1, wherein said tissue comprises tumor tissue.

4. The electrosurgical instrument of claim 3, wherein said thermal treatment results in tumor destruction, lesion formation, or the denaturization, dessication, coagulation, or ablation of tumor tissue.

5. The electrosurgical instrument of claim 1, wherein said instrument is monopolar.

6. The electrosurgical instrument of claim 1, wherein said instrument is bipolar.

7. The electrosurgical instrument of claim 1, wherein the intermediate portion of said floating electrode comprises an elongate hollow cylinder.

8. The electrosurgical instrument of claim 1, wherein said floating electrode comprises an elongate hollow cylinder of constant diameter.

9. The electrosurgical instrument of claim 8, wherein said one or more perforations are distributed about the periphery of said elongate hollow cylinder.

10. The electrosurgical instrument of claim 8, wherein said one or more perforations are provided only on a top surface of said elongate hollow cylinder.

11. The electrosurgical instrument of claim 8, wherein said one or more perforations comprise a first set of perforations on a top surface of said elongate hollow cylinder and a second set of perforations on a bottom surface of said elongate hollow cylinder.

12. The electrosurgical instrument of claim 11, wherein said first and second sets of perforations are laterally offset.

13. The electrosurgical instrument of claim 1, wherein said floating electrode is relatively tubular, further wherein a diameter of said floating electrode tapers at a neck point from a relatively widened tubular proximal portion to a relatively narrowed tubular intermediate portion.

14. The electrosurgical instrument of claim 1, wherein the exposed distal end of said active electrode is proximal to the most proximal of said one or more perforations provided along the lengths of said intermediate portion.

15. The electrosurgical instrument of claim 1, wherein the exposed distal end of said active electrode extends distally beyond said neck point into said intermediate portion.

16. The electrosurgical instrument of claim 1, wherein the exposed distal end of said active electrode extends distally beyond the most proximal of said one or more perforations provided along the lengths of said intermediate portion.

17. The electrosurgical instrument of claim 1, wherein said intermediate portion comprises first and second parallel planar surfaces.

18. The electrosurgical instrument of claim 1, wherein said one or more perforations are provided only on the first parallel planar surface.

19. The electrosurgical instrument of claim 1, wherein said one or more perforations comprise a first set of perforations on said first planar and a second set of perforations on said second planar surface.

20. The electrosurgical instrument of claim 1, wherein said first and second sets of perforations are laterally offset.

21. The electrosurgical instrument of claim 1, wherein said active electrode is powered by radio-frequency.

22. The electrosurgical instrument of claim 1, wherein said an active electrode comprises an elongate solid metal rod or filament disposed within said irrigation lumen.

23. The electrosurgical instrument of claim 1, wherein said an active electrode comprises a metal tube that is contiguous with said at least one irrigation lumen.

24. The electrosurgical instrument of claim 1, wherein the distal end of said active electrode comprises a solid metal rod or filament that extends in a distal direction into said steam generating cavity so as enhance boiling of said irrigant.

25. The electrosurgical instrument of claim 1, wherein said electrode assembly further comprises a tubular dielectric member disposed about said active electrode and encapsulating said exposed distal tip so as to define a channel in fluid communication with said steam generating cavity via one or more ports disposed in a wall of said tubular dielectric member.

26. The electrosurgical instrument of claim 1, wherein said at least one irrigation lumen comprises a single linear tube.

27. The electrosurgical instrument of claim 1, wherein said at least one irrigation lumen comprises a plurality of discrete channels arrayed in an annular fashion about the active electrode.

28. The electrosurgical instrument of claim 1, wherein said active electrode is powered by radio-frequency.

29. The electrosurgical instrument of claim 1, wherein said distal element is made from a dielectric material.

30. The electrosurgical instrument of claim 1, wherein said distal element is made from a conductive material.

31. The electrosurgical instrument of claim 30, wherein said conductive distal element is integral with said floating electrode

32. The electrosurgical instrument of claim 1, wherein said distal element comprises a conical tip that is sufficiently sharp to permit insertion of said electrode assembly into said target tissue.

33. The electrosurgical instrument of claim 1, wherein said distal element comprises a rounded tip.

34. The electrosurgical instrument of claim 1, wherein said distal element is wedge-shaped.
